(19) United States
(12) Patent Application Publication
(10) Pub. No.: US 2023/0075871 A1
(43) Pub. Date: Mar. 9, 2023

(54) METHOD FOR DETERMINING RADIATION EXPOSURE WITH SENSITIVE AND SPECIFIC GENE EXPRESSION SIGNATURES

(71) Applicants: Peter Keith Rogan, London (CA); Eliseos J. Mucaki, London (CA)

(72) Inventors: Peter Keith Rogan, London (CA); Eliseos J. Mucaki, London (CA)

(21) Appl. No.: 17/402,550

(22) Filed: Aug. 15, 2021

(51) Int. Cl. G16B 20/00; C12Q 1/6883

(52) U.S. Cl. CPC G16B 20/00; C12Q 1/6883; C12Q 2600/158

(57) ABSTRACT

The present invention discloses a method for determining improved radiation gene expression profiles by sequential application of sensitive and specific gene signatures. The method involves evaluating a sample of target cells from a patient against a highly sensitive, first radiation gene signature, to determine the radiation exposed gene signature. If the signature does not completely distinguish radiation exposures from other conditions or phenotypes, the sample may be evaluated against a second radiation gene signature, which is a radiation gene signature with high specificity. On sequential application of sensitive and specific gene signatures, any misclassified unirradiated samples remaining in the determined gene signatures are identified and removed. Thus, the method enables rejection of radiation signatures with high false positive radiation diagnosis in conditions that confound the results with the first signature. The method derives individual or sequential sensitive and specific radiation signatures with low misclassification rates due to confounding phenotypes, in either controls and test samples.

### TECHNICAL FIELD

The present disclosure relates generally to radiation gene expression profiles, and more particularly, to a method for determining ionizing radiation-exposed gene expression profiles by sequentially applying, sensitive and specific gene signatures to samples with unknown levels of exposure.

### Supplemental Data

The following list of data tables were submitted electronically as text files via the USPTO Electronic Filing System (EFS) and are hereby incorporated by reference. Numerals in column headings, such as “Model 1” refer to footnote 1. Data fields generally have the format of a percentage followed by a number in parentheses or a percentage followed by a number in parentheses further followed by a footnote number. A data field entry such as “−4” indicates no data with reference to footnote 4. Some data values, such as “Mutual Information” are indicated as a decimal number. Genes are identified by gene name, such as TRIM24.

| Table | File Name |
|---|---|
| Supplemental Table S1: mRMR Rankings of | Table_S1 |
| Radiation Response Genes in Ex-Vivo Control | |
| and Radiation Exposed Paired Datasets | |
| Supplemental Table S2A: Influenza and | Table_S2A |
| Dengue Infection Increase False Positives by | |
| Radiation Gene Signatures | |
| Supplemental Table S2B: Infectious, Inherited | Table_S2B |
| and Non-Inherited Blood-borne Disorders | |
| Increase False Positives by Radiation Gene | |
| Signatures | |
| Supplemental Table S3A: False Positive Rate | Table_S3A |
| after Feature Removal of M1-M4 Against | |
| Blood Disease Pathologies | |
| Supplemental Table S3B: False Positive Rate | Table_S3B |
| after Feature Removal of KM3-KM7 Against | |
| Blood Disease Pathologies | |
| Supplemental Table S4A: Radiation | Table_S4A |
| Signatures derived from GSE102971 with | |
| Increased FPs evaluating Blood Disease | |
| Pathologies | |
| Supplemental Table S4B: Radiation | Table_S4B |
| Signatures derived from GSE85570 with | |
| Increased FPs evaluating Blood Disease | |
| Pathologies | |
| Supplemental Table S4C: Radiation | Table_S4C |
| Signatures derived from GSE6874 with | |
| Increased FPs evaluating Blood Disease | |
| Pathologies | |
| Supplemental Table S4D: Radiation | Table_S4D |
| Signatures derived from GSE10640 with | |
| Increased FPs evaluating Diseased Blood | |
| Disease Pathologies | |
| Supplemental Table S5A: Feature Removal | Table_S5A |
| Analysis Gene Signatures derived from | |
| Radiation Dataset GSE102971 | |
| Supplemental Table S5B: Feature Removal | Table_S5B |
| Analysis Gene Signatures derived from | |
| Radiation Dataset GSE85570 | |
| Supplemental Table S6A: Derivation of | Table_S6A |
| Radiation Models based on Secreted Genes | |
| Table S6B: Testing Secreted Gene Radiation | Table_S6B |
| Signatures against Blood-Borne Diseased | |
| Patients | |
| Supplementary Table S6C: Normalized | Table_S6C |
| change in mRNA expression of plasma protein | |
| encoded gene signature components after | |
| Radiation Exposure | |
| Supplemental Table S6D: Testing Secreted | Table_S6D |
| Gene Radiation Signatures against Influenza | |
| and Dengue Infection | |

LENGTHY TABLES
The patent application contains a lengthy table section. A copy of the table is available in electronic form from the USPTO web site (https://seqdata.uspto.gov/?pageRequest=docDetail&DocID=US20230075871A1). An electronic copy of the table will also be available from the USPTO upon request and payment of the fee set forth in 37 CFR 1.19(b)(3).

### BACKGROUND

Potential ionizing radiation exposures from environmental exposures including industrial nuclear accidents, military incidents, or terrorism are also threats to public health. There is a need for large scale biodosimetry testing, which requires efficient screening techniques to differentiate exposed individuals from non-exposed individuals and to determine the severity of exposure. Ionizing radiation is also used in biomedical diagnostic and therapeutic applications, where it may be used to monitor and calibrate absorbed radiation levels. One biodosimetric method involves using machine learning (ML) to derive radiation signatures from genomic, transcriptomic, and metabolomic data to diagnose radiation exposure of persons.

When an individual is irradiated by ionizing radiation, a subset of genes in cells are either activated or repressed. Transcription of these genes is altered. Changes occur in the levels of single stranded (SS) coding mRNAs (which are translated into proteins) or miRNAs that function in the overall biological response to radiation at the cellular level. Methods of quantification of mRNA levels in response to a wide variety of external stimuli, of which radiation is a notable example, are well known in the art. The degree to which single-stranded oligonucleotides selected for specific genes hybridize to specific, labelled SS radiation-responsive transcripts determines the steady-state level of expression of that mRNA in radiation-exposed cells. These changes are dynamic over time, however prodromal clinical symptoms are associated with changes in expression generally during the initial 72 hours post-radiation exposure. The amount of labelled mRNA in a sample is used to quantitate the amount of this molecule or radiation gene expression profiles. Differences in the level of expression of mRNA from the same gene in controls, i.e. unexposed cells, indicate the gene is responsive to radiation exposure, or to other conditions. These measurements are made for each of the genes that comprise a radiation signature, and the set of these measurements are grouped for each sample in which radiation exposure is to be determined.

The general approach that determines the selection of genes and measures the levels of mRNA that define gene expression signatures of radiation exposure is explained as follows. In gene expression microarrays or reverse-transcription of mRNA coupled to the polymerase chain reaction, one or more nucleic acid probes for gene(s) of interest are hybridized to RNA isolated from cells, preferably obtained from blood, but other tissues may also be analyzed (Zhao et al. 2018a). The extent of hybridization probes is used to quantitate how much transcription of the gene(s) of interest has occurred. Alternatively, quantification of cDNA synthesized from RNA can be obtained from sequences of the transcriptome and by determining the normalized count of transcripts from each of these genes. With radiation exposure, numerous genes are induced or repressed after exposure. Exposure has typically been inferred from subsets of genes that are activated or repressed, specifically from the set of genes whose expression levels significantly change in response to radiation, which are considered to be candidates comprising the gene signature. Once an optimal set of radiation genes candidates has been compiled, this gene combination or signature can be used as input for machine learning methods that derive the relative contributions that each gene makes towards the decision to classify the individual as either exposed or unexposed. This signature is then used to evaluate individuals with suspected exposures.

Expression levels of signature genes in an RNA sample of cells from such individuals are quantified, and the quantities are input into the classifier, which determines whether the sample(s) have been exposed to radiation.

However, genes expressed or repressed in response to radiation are also altered by other conditions which share clinical sequelae of acute radiation syndrome at the prodromal phase, such as Influenza and Dengue viral infections (Rogan et al. 2021). Similarity between the clinical presentation between these conditions motivated investigation into whether radiation-derived gene expression signatures could differentiate radiation exposures from these other conditions. The expression of several genes present in radiation signatures exhibited similar changes in these other blood-borne infections. Therefore, methods are needed to refine the radiation gene profiles so that radiation exposures are detected unequivocally, by eliminating false positive (FP) classifications of samples that instead result of blood-borne “confounding conditions”, such as Influenza A or Dengue infection. These confounders, if not detected or eliminated, can reduce the overall accuracy of radiation signatures. Therefore, the instant invention presents a method to identify and eliminate misclassified samples with underlying hematological or infectious conditions, leaving only samples with true radiation exposures.

### SUMMARY

The present invention discloses a method for improving the overall performance and accuracy of radiation gene expression profiles which identify exposed individuals based on changes in the expression of genes that respond to ionizing radiation stimuli. The method sequentially applies different gene signatures that are respectively optimized for maximizing sensitivity and specificity of gene signatures. The method is configured to identify and eliminate misclassified samples with underlying hematological or infectious conditions, leaving only samples with true radiation exposures. The method significantly improves the diagnostic accuracy by selecting genes that maximize both sensitivity and specificity in the appropriate tissue using combinations of the best signatures for each of these classes of signatures.

In one embodiment, the method for determining radiation gene expression profiles is disclosed. At one step, a sample of target cells from an exposed individual is provided. At another step, the sample is evaluated against a first gene signature. First, gene signatures are derived from a set of samples of known radiation exposures that are distinguished from unexposed samples. In one embodiment, the first signature is a gene expression signature that is highly sensitive for exposure to radiation. For the purposes of this disclosure a signature is considered to be highly sensitive if it diagnoses an irradiated sample with greater than 80% accuracy. However, an accuracy of at least 90% is preferable. When this gene expression signature also specifically distinguishes actual radiation exposures from confounding condition(s), then no additional analyses or signature evaluation are needed. However, few gene signatures satisfy the requisite criteria of having both high sensitivity and high specificity for radiation exposure. Thus, another step is usually required after analysis with a sensitive signature to unequivocally determine if the changes in gene expression in the sample are radiation-induced. At yet another step, the sample is evaluated against a second gene signature to confirm radiation exposure indicated by the first signature. In one embodiment, the second gene signature is a radiation gene signature with high specificity. At yet another step, any misclassified and unirradiated samples erroneously classified as radiation-exposed by the first gene signature are identified as unirradiated using the second gene signature, and then reclassified as unirradiated. Thus, the method facilitates rejection of radiation signatures with high false positive radiation diagnosis in confounding conditions and derivation of radiation signatures with low misclassification rates in confounders in both controls and test samples. The method mitigates false positive predictions due to similar expression patterns caused by confounding conditions through sequential evaluation of gene expression levels in samples using both the first high sensitivity gene signature and the second high specificity gene signature.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 summarizes a method of evaluation of disease conditions that confound radiation gene expression signatures, according to an embodiment of the present invention. The traditional validation approach was used to evaluate unirradiated datasets for hematological conditions to assess the performance of radiation gene signatures derived in Zhao et al. (2018a). With this approach, machine learning models of gene signatures with high rates of false positive (FP) radiation diagnosis can be identified and rejected due to confounding conditions while identifying which confounders make individuals ineligible for a radiation gene signature assay with these models, In these cases, new radiation gene signatures are required that improve detection of FPs in both controls and test subjects.

FIG. 2 is a schematic diagram illustrating sequential application of sensitive and specific gene signatures to identify radiation exposed samples. False positive predictions due to differential expression caused by confounding conditions could be mitigated by following a sequential approach where samples are evaluated with both a highly sensitive radiation gene signature and a second signature with high specificity. M4, for example, is highly sensitive when validated against radiation dataset GSE10640[GPL6522] (88% accuracy), where all incorrect classifications were due to FP predictions (zero false negatives [FN]). Predicted irradiated samples could then be evaluated with a highly specific model such as SM3, which would identify and remove any misclassified unirradiated samples remaining in the set and leave only TPs.

FIG. 3A-FIG. 3D illustrates graphs which show the performance of traditionally-validated radiation signatures on confounding disease samples after categorical stratification by subphenotypes, according to an embodiment of the present invention. Sankey diagrams delineate the numbers of diseased individuals and controls were properly (TN; true negative) and improperly classified (FP; false positive) by a radiation gene signature. Panel 3A: The radiation signature M4 incorrectly classified 53% of dengue-infected patient as irradiated, however all convalescent patients were properly classified (0% FP). Panel 3B: Similarly, the FP rate of M1 decreased considerably after patient recovery (27% FP rate [N=19] against samples <3 days after symptoms; 3% [N=2] after 2-5 weeks). Panel 3C: The FP rate of M3 was higher for severe malarial anemia patients versus those with cerebral malaria, suggesting that the differential expression caused by the two infection types may diverge in such a way that is measurable by M3, Panel 3D: Conversely, the FP rate of venous thromboembolism patients by M4 was not influenced on whether the disease was recurrent.

FIG. 4A-FIG. 4D are graphs illustrating the percentage of samples misclassified as irradiated, according to an embodiment of the present invention. Radiation gene signatures M1-M4 and KM3-KM7 were accurate when predicting radiation exposure (presented in order of descending accuracy and grouped by validation type in panels 4A and 4B), However, many of these models falsely predicted individuals with blood-borne disorders (thromboembolism [4A] and sickle cell disease [4C]) and infectious diseases (S. aureus [4B] and malaria [4D]) as irradiated (% FP provided for individuals with the indicated disease [hatched; top], and controls [clear; bottom]). In general, the FP rate was high for all traditional validated (M1-M4) and most k-fold validated models (KM4, KM6 and KM7). Models KM3 and KM5 had a low FP rate across all datasets tested.

FIG. 5A-FIG. 5B are graphs illustrating DDB2 and BCL2 expression distributions of different samples from hematological disorder and radiation-exposure datasets, according to an embodiment of the present invention. Normalized distribution of gene expression of confounder datasets (VTE: Venous Thromboembolism [orange]; SAu: S. aureus [teal]; Sic: Sickle Cell [yellow]; and Mal: Malaria [dark green]) for the genes A) DDB2 and B) BCL2 are presented as violin plots, where the expression of individuals with these conditions are divided by those predicted as irradiated (FP; left) or unirradiated (TN; right) by signature M4. Control expression of radiation-exposed (Irr.) and (Non) unirradiated individuals are indicated by distributions labeled with light (GSE6874) and dark (GSE10640) red outlines on the right side of each panel. Significant expression differences between FP and TN samples from the same dataset predicted with signature M4 are indicated by brackets above the corresponding pair of predictions.

FIG. 6A-FIG. 6B are graphs illustrating the effects of removing multiple genes from gene signatures in terms of their contribution and misclassification rate of confounding datasets, according to an embodiment of the present invention. Accuracy of M4 was significantly influenced by hematological confounders such as venous thromboembolism (6A) and S. aureus infection (6B). M4 misclassified diseased individuals (circles) far more often than controls (squares). Feature removal analysis of M4 determines if a particular gene was contributing to the FP rate by observing how accuracy changes when a gene is removed. While M4 accuracy improved with the removal of PRKDC, IL2RB and LCN2, no individual gene restored misclassification back to control levels suggesting multiple genes contribute to the confounding effects of these diseases.

FIG. 7A-FIG. 7D illustrates graphs of secretome-derived gene signatures that reduce misclassification of confounder phenotypes, according to an embodiment of the present invention. Radiation signatures which consist exclusively of genes encoding plasma secreted proteins were derived following the same basic approach of Zhao et al. (2018a). These models showed generally favorable performance when tested against an independent radiation dataset by k-fold validation (grey bar; in order of descending accuracy). Five secretome radiation signatures were derived consisting of 7-75 genes (SM1-SM5). Two models (SM3 and SM5) show high specificity across all hematological conditions tested.

FIGS. 8-FIG. 10 shows expression levels and thresholds of DDB2, IL2RB, PCNA and PRKDC for 3 individuals with thromboembolism (Dataset GEO: GSE19151) predicted to be irradiated by signature M4 (GSM474819, GSM474822, and GSM474828 identify the individuals in this dataset)

### DETAILED DESCRIPTION OF THE INVENTION AND EXAMPLE EMBODIMENTS

The present invention discloses a method for determining improved radiation gene expression profiles by sequential application of sensitive and specific gene signatures. The method involves steps of: providing a sample of target cells from a patient; evaluating the sample against a highly sensitive first gene signature; detecting radiation exposed gene signatures; evaluating the sample against a second gene signature with high specificity, and identifying and removing any misclassified unirradiated samples remaining in the detected radiation exposed gene signatures.

### Datasets Evaluated

A series of highly accurate radiation gene signatures were derived in Zhao et al. (2018a). When these models were tested against non-irradiated individuals with viral infections (Influenza A and Dengue), a significant proportion were incorrectly classified as irradiated (Rogan et al. 2020). We explored whether other blood-borne diseases (infections in the blood or inherited and non-inherited hematological disorders) can also confound these signatures utilizing public gene expression datasets, and derived new radiation datasets in order to investigate whether this is an issue inherent to signatures with alternate gene compositions

Inclusion for any gene in a ML-based signature required expression data to be present in both training and validation radiation datasets (Gene Expression Omnibus (GEO; https://www.ncbi.nlm.nih.gov/geo) database identifiers: GSE701 [Jen and Cheung, 2003], GSE1725 [Rieger et al. 2004], GSE6874[GPL4782; Dressman et al. 2007] and GSE10640[GPL6522; Meadows et al. 2008]). As a consequence, several well-known radiation genes which have appeared in other radiation gene signatures (Paul and Amundson, 2008; Oh et al. 2014; Port et al. 2017; Tichy et al. 2018; Jacobs et al. 2020) were previously not considered and thus not present in any of the derived models. Genes were excluded either because: 1) it was absent from one or more datasets (e.g. FDXR, RPS27L, AEN were absent from the GSE10640[GPL6522] dataset); 2) they was mislabelled in the dataset with a legacy name leading to a mismatch between datasets (e.g. PARP1 appears as ADPRT in GSE6874[GPL4782]); 3) all available probes detected a derived secondary RNA, such as a microRNA or lncRNA (e.g. BBC3 probes also detected multiple microRNAs in microarray used in GSE1725; POU2AF1 probes in GSE701 also labelled 10C101928620′); or 4) it was missing from the set of curated radiation response genes (Zhao et al. 2018a; e.g. PHPT1, VWCE, WNT3). Imputation of expression from nearest neighbours, which is intended to replace missing data from small numbers of patients (<5%), could not overcome this limitation.

To address missing genes in radiation response signatures (Zhao et al. 2018a), we attempted development of novel signatures based on more recent irradiated blood studies, including GEO: GSE26835, GSE85570, GSE102971 and ArrayExpress (https://www.ebi.ac.uk/arrayexpress) database: E-TABM-90. GSE85570 consisted of pre-treatment blood from 200 prostate cancer patients, with half of each sample receiving 2Gy of radiation, followed by analysis on HT HG-U133+ PM microarrays (Affymetrix). In GSE102971, peripheral blood (PB) from healthy volunteers was irradiated ex-vivo at 2, 5, 6 and 7 Gy and analyzed 24 hours after exposure with a custom commercial 4×44K human microarray (Agilent). In E-TABM-90, ex-vivo lymphocytes from 50 prostate cancer patients (2 years post-radiotherapy) received either 2 Gy radiation or were unirradiated, and RNA was analyzed on HG-U133A microarrays (Affymetrix). In GSE26835, immortalized lymphoblastoid cells at 2 and 6 hours post 10Gy of radiation were analyzed on U133A microarrays. Previous radiation gene signatures (Zhao et al. 2018a) were derived from GSE6874[GPL4782] and GSE10640[GPL6522] 6 hours post-exposure from healthy donors and patients undergoing total body radiation (˜2 Gy), and then analyzed with non-commercial, custom microarrays.

To investigate whether other disorders and phenotypes—besides Influenza and Dengue fever infected patients—could also confound radiation signatures, we assessed performance of radiation signatures utilized in this study with available gene expression datasets for other blood-borne diseases. These datasets include GEO: GSE117613 (Cerebral Malaria and Severe Malarial Anemia; Nallandhighal et al. 2019), GSE35007 (Sickle cell disease [SCD] in children; Quinlan et al. 2014), GSE47018 (Polycythemia Vera; Spivak et al. 2014), GSE19151 (single and recurrent venous thromboembolism; Lewis et al. 2011), GSE30119 (Staphylococcus [S.] aureus infection; Banchereau et al. 2012), and GSE16334 (aplastic anemia; Vanderwerf et al. 2009). Other haematological datasets were also considered for evaluation as potential confounders, however too few samples in these datasets were available (<10) to determine accurate classification rates. Thus, the datasets evaluated in the instant should not be considered to be a comprehensive set of potential confounders. Rather, the range of phenotypes and transcriptional responses encompassed by these conditions are examples of their broad impact on radiation response. They represent the minimum spectrum of potential blood-borne phenotypes that could confound responses to radiation exposure using the gene signatures obtained with the instant method.

### Data Pre-Processing

Microarray data downloaded from GEO datasets GSE85570 and GSE102971 were pre-processed as described (Zhao et al. 2018a). Briefly, missing gene expression values were imputed (or removed if gene is <95% complete) by nearest neighbours, the expression of patient replicates was averaged, and gene expression was z-score normalized. Gene expression was analyzed previously implicated in the radiation response (N=998), plus 13 additional radiation genes that were described in other studies, including CD177, DAGLA, HIST1H2BD, MAMDC4, PHPT1, PLA2G16, PRF1, SLC4A11, STAT4, VWCE, WLS, WNT3, and ZNF541 (N=1,011 genes total).

### Derivation of Radiation Gene Signatures

mRMR was performed against the expression of the radiation gene subset and assigned a rank to each gene in accordance to the mutual information difference (MID) criterion (Ding and Peng; 2005; Mucaki et al. 2016). Briefly, mRMR first selects the genes with the highest mutual information (MI) with radiation, then corrects for redundancy by selecting the gene candidate with the highest difference between the mutual information and the mean mutual information between all previously selected genes with the candidate gene as a probability vector. The second (and some subsequent) selected gene(s) tend to exhibit lower mutual information with radiation, but has a non-redundant expression pattern relative to the preceding gene(s); nevertheless, higher ranked genes exhibit greater MI then those with lower ranking. Minimization of redundancy can result in some genes with low MI values being assigned high ranks. While the second and other lower ranked genes met this criterion, they could exhibit low MI values, consistent with weak radiation response. Gene rankings by mRMR and the computed mutual information for each radiation gene in each of the datasets evaluated.

Support Vector Machine (SVM)-based gene signatures were derived with greedy feature selection methods, including Forward sequential feature selection (FSFS), backward sequential feature selection (BSFS) and complete sequential feature selection (CSFS; Zhao et al. 2018a). Software is provided in a Zenodo archive (Zhao et al. 2018b). Both FSFS and BSFS models were derived from the top 50 ranked mRMR genes, in addition to the following previously described radiation responsive genes: AEN, BAX, BCL2, DDB2, FDXR, PCNA, POU2AF1, and WNT3. SVMs were derived with a Gaussian radial basis function kernel by iterating of box-constraint (C) and kernel-scale (a) parameters and gene features, minimizing to either misclassification or log loss (the similarity of predicted outcomes to the ground truth; Zhao et al. 2018a; Bagchee-Clark et al. 2020). Performance was assessed by applying these gene signatures to a validation dataset and evaluated based on misclassification rates, log loss, Matthews correlation coefficient, or goodness of fit. We report misclassification rates in order to simplify comparisons of results between radiation exposed and disease confounder datasets. Signatures with high misclassification rates in radiation validation datasets (>50%) were not reported. Radiation gene signatures derived from different datasets can be composed of different gene combinations. This can be attributed to many factors, including distinct microarray platforms, batch effects, and inter-individual variation in the expression of genes which cannot be fully addressed by data normalization. These differences contribute to MI variability, which both alters mRMR rank and influences gene selection by feature selection.

We assessed expression dataset quality based on the dynamic responses of mRMR genes to radiation exposure, since potential confounders could potentially also alter aspects of these responses. MI between gene expression and radiation dose was determined for the four radiation datasets evaluated. Referring to Table 2, datasets GSE85570 and GSE102971 both showed high MI with radiation exposure (maximum MI>0.7 bits for both datasets; 77 and 115 genes with >0.2 bits MI, respectively). Datasets E-TABM-90 and GSE26835 failed to fulfill quality control criteria and were not considered further. Both exhibited low MI for top ranked genes with radiation exposure, relative to their rankings in GSE85570 and GSE102971. Of the top 50 ranked genes in E-TABM-90, 13 genes had MI values <10% of the MI of the top ranked gene [0.3 bits], and 856 of 860 genes in the complete dataset had MI<0.2 bits. The maximum MI for GSE26835 was 0.25 bits, 40 out of 50 top ranked genes exhibited <10% MI of the maximum, and the radiation response genes DDB2, PCNA, FDXR, AEN, and BAX, had unexpectedly low rankings (>100; 2 h and 6 h post-exposure) and MI<0.15 bits. The Low MIs across all eligible genes indicates that the response to radiation was nearly random. Radiation toxicity in E-TABM-90 and cell line immortalization in GSE26835 appears compromise their radiation response.

TABLE 1
Traditional and K-Fold Validated Radiation Gene Expression Signature
Derived from Radiation Dataset GSE1725

| | |
|---|---|
| KM1 | GADD45A DDB2 |
| KM2 | PPM1D DDB2 CCNF CDKN1A PCNA GADD45A PRKAB1 TOB1 |
| | TNFRSF10B MYC CCNB2 PTP4A1 BAX CCNA2 ATF3 LIG1 CCNG1 |
| | FHL2 PPP1R2 MBD4 RASGRP2 UBC NINJ1 TRIM22 IL2RB TP53BP1 |
| | PTPRCAP EEF1D PTPRE RAD23B EIF2B4 STX11 PTPN6 STK10 |
| | PSMD1 BTG3 MLH1 RNPEP HSPD1 UNG PTPRC PTPRA BCL2 GSS |
| | SH3BP5 TPP2 IDH3B CCNH STK11 EIF4EBP2 HSPA4 FADS2 RPA3 |
| | GZMK ANXA4 ICAM1 PPID LMO2 PPIE NUDT1 FUS POLR2A LY9 |
| | RPA1 PTS TNFRSF4 RPA2 PSMD8 GCDH MAN2C1 PTPN2 RUVBL1 |
| | ATP5H GK CD79B MAP4K4 POLE3 PRKCH AKT2 MOAP1 CCNG2 |
| | ALDOA SRD5A1 HAT1 XRCC1 EIF2S3 RAD1 UBE2A ZFP36L1 CD8A |
| | TALDO1 GPX4 SSBP2 ERCC3 ATP5O PEPD EIF4G2 ACO2 HEXB |
| | UBE3A ARPC1A PSMD10 PRCP PPIB ZNF337 CETN2 RPL29 |

Derived from Radiation Dataset GSE10640[GPL6522]

| | |
|---|---|
| M1 | DDB2 HSPD1 MAP4K4 GTF3A PCNA MDH2 |
| M2 | DDB2 GTF3A TNFRSF10B |
| KM3 | DDB2 RAD17 PSMD9 LY9 PPIH PCNA MDH2 MOAP1 TP53BP1 |
| | PPM1D ATP5G1 BCL2L2 ENO2 PTP4A1 PSMD8 LIG1 FDPS OGDH |
| | CCNG1 PSMD1 |
| KM4 | DDB2 HSPD1 ICAM1 PTP4A1 GTF3A LY9 |
| KM5 | RAD17 TNFRSF10B PSMD9 LY9 PPIH PCNA ZNF337 MDH2 |
| | TP53BP1 PPM1D ZFP36L1 ATP5G1 ALDOA BCL2L2 ENO2 GADD45A |
| | PTP4A1 PSMD8 LIG1 ATP5O FDPS OGDH PSMD1 |

Derived from Radiation Dataset GSE6874[GPL4782]

| | |
|---|---|
| M3 | DDB2 CD8A TALDO1 PCNA EIF4G2 LCN2 CDKN1A PRKCH ENO1 |
| | PPM1D |
| M4 | DDB2 CD8A TALDO1 PCNA LCN2 CDKN1A PRKCH ENO1 GTF3A |
| | IL2RB NINJ1 BAX TRIM22 PRKDC GADD45A MOAP1 ARPC1B LY9 |
| | LMO2 STX11 TPP2 CCNG1 GABARAP BCL2 GSS FTH1 |
| KM6 | DDB2 PRKDC PRKCH IGJ |
| KM7 | DDB2 PRKDC TPP2 PTPRE GADD45A |

Performance metrics for these radiation gene signatures are available in Zhao et al. (2018a) Tables 2 (k-fold validated signatures [KM1-KM7]) and 3 (traditionally validated signatures [M1-M4]);
FS—Feature Selection metrics

Signatures M1-M4 and KM1-KM7

TABLE 2
Radiation Gene Expression Signatures derived from GSE85570 and GSE102791 Datasets

| | FS. | FS. | FS. | % FP by Confounder (Disease/Controls) | | | |
|---|---|---|---|---|---|---|---|
| Signature (C, σ) | Algorithm | Misclass | Log Loss | Thrombosis | S. Aureus | SCD | Malaria |

a) Derived from Radiation Dataset GSE102791

| | | | | | | | | |
|---|---|---|---|---|---|---|---|---|
| M5 | AEN, BCL2 | FSFS* | — | 8.1E−15 | 0.57/0.40 | 0.48/0.41 | 0.49/0.43 | 0.41/0.08 |
| | (100000, | | | | | | | |
| | 10000) | | | | | | | |
| M6 | RPS27L, | FSFS | — | 5.2E−15 | 0.78/0.69 | 0.49/0.46 | 0.50/0.43 | 0.71/0.25 |
| | ZMAT3 | | | | | | | |
| | (10, 10) | | | | | | | |
| M7 | AEN, | CSFS | 0% | — | 0.60/0.83 | 0.71/0.66 | 0.66/0.72 | 0.68/0.42 |
| | ERCC1, | | | | | | | |
| | BAX (1000, | | | | | | | |
| | 100) | | | | | | | |
| M8 | AEN, | FSFS | — | 5.2E−15 | 0.00/0.00 | 0.00/0.00 | 0.56/0.26 | 1.00/1.00 |
| | TNFRSF10B | | | | | | | |
| | (100, 100) | | | | | | | |

b) Derived from Radiation Dataset GSE85570

| | | | | | | | | |
|---|---|---|---|---|---|---|---|---|
| M9 | BAX, FDXR | FSFS | 0% | — | 0.64/0.27 | 0.46/0.64 | 0.52/0.33 | 0.44/0.75 |
| | (1000, 1) | | | | | | | |
| M10 | BAX, FDXR, | FSFS | 0% | — | 0.80/0.46 | 0.84/0.65 | 0.67/0.69 | 0.68/1.00 |
| | XPC (1000, | | | | | | | |
| | 10) | | | | | | | |
| M11 | BAX, DDB2 | FSFS | 0% | — | 0.34/0.76 | 0.52/0.48 | 0.48/0.49 | 0.59/0.17 |
| | (100, 10) | | | | | | | |
| M12 | BAX, DDB2, | FSFS | 0% | — | 0.51/0.31 | 0.40/0.23 | 0.41/0.47 | 0.44/0.20 |
| | SLC7A6 (10, | | | | | | | |
| | 10) | | | | | | | |
| M13 | RPS27L, | FSFS | 0% | — | 0.77/0.40 | 0.63/0.55 | 0.54/0.67 | 0.68/0.25 |
| | DDB2, | | | | | | | |
| | ARL6IP1, | | | | | | | |
| | TRIM32 | | | | | | | |
| | (100000, 1) | | | | | | | |

*Models derived using 0Gy, 2Gy and 5Gy samples only (excludes 6Gy and 7Gy samples from GSE102791)

### Derivation of Secretome Gene Signatures

Radiation responses encompass global protein synthesis which significantly increase 4-8 hr after initial exposure (Braunstein et al. 2009), with some profile changes detectable weeks to months later (Pernot et al. 2012; Hall et al. 2017). Radiation signatures in blood have been derived from proteins secreted in plasma (Wang et al. 2020) and expressed by multiple cell lineages (Ostheim et al. 2021). If radiation causes gene signature mRNAs encoding components of the plasma secretome to exhibit short term changes in abundance that are reflected in monotonic, codirectional changes in plasma protein concentration, then mRNA levels could be used as a surrogate for changes in plasma protein levels. Significant correlations between mRNA and protein expression have been shown when the data have been transformed to normal distributions (Greenbaum et al. 2001; Greenbaum et al. 2002). This is the approach that we have taken in deriving mRNA signatures of ionizing radiation response from the secretome.

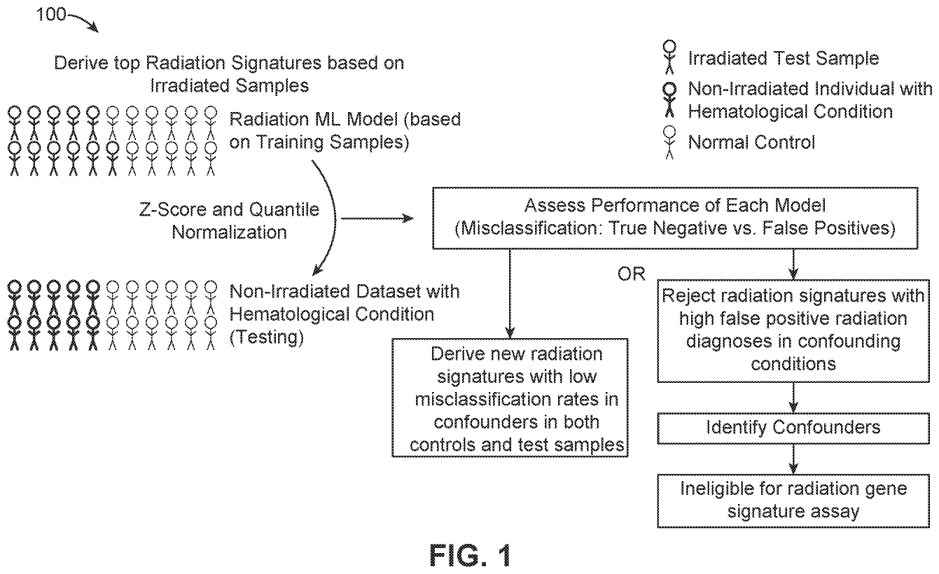

Only genes whose products are expressed blood plasma were used to derive gene expression signatures by biochemically inspired machine learning, as shown in FIG. 1). To derive this initial set of proteins, the Human Protein Atlas “Human Secretome” [http://www.proteinatlas.org/humanproteome/secretome] and the Plasma Protein [http://www.plasmaproteomedatabase.org] Databases were cross-referenced to create a list of 1377 shared genes. We then utilized the Genotype-Tissue Expression (GTEx) Portal (https://gtexportal.org/home/) to identify which of the genes encoding these secreted proteins are expressed in either leukocytes or transformed lymphoblasts (where Transcripts Per Million (TPM)>1; N=682). We identified these remaining genes when present in two relevant radiation-dose expression datasets GSE6874[GPL4782] (N=428) and GSE10640[GPL6522] (N=325). These genes were then used to develop ML models of genes encoding for human plasma proteins by CSFS, BSFS, and FSFS, in which samples were exposed to ionizing radiation.

The following detailed descriptions of FIGS. 1 through 7 summarize the preceding procedures and illustrate example results obtained with the instant invention:

FIG. 1 is a method (100) of evaluation of disease conditions that confound the results of radiation gene signatures, according to an embodiment of the present invention. The non-irradiated datasets with hematological conditions and radiation machine learning model are used to assess the performance of radiation gene signatures. The gene signature models with high false positive rate (FP) radiation diagnoses in confounding conditions are rejected. This identifies the confounders that affect the eligibility of individuals for a radiation gene expression signature assay. For individuals with these conditions, new radiation gene signatures are required that show improved FP rates in both controls and test subjects.

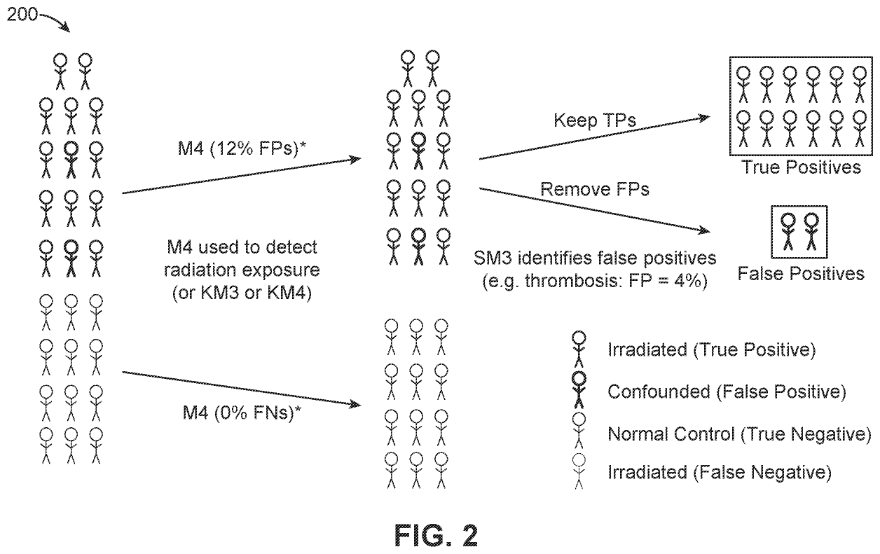

FIG. 2 is a schematic diagram (200) illustrating sequential application of sensitive and specific gene signatures to identify radiation exposed gene signatures. The false positive predictions due to differential expression caused by confounding conditions is mitigated by a sequential approach where samples are evaluated with both a highly sensitive radiation gene signature first, and a signature with high specificity second. Signature M4 (Table 1), for example, is highly sensitive when validated against radiation dataset GSE10640[GPL6522] (88% accuracy), where all incorrect classifications were due to FP predictions (zero false negatives [FN]). Predicted irradiated samples could then be evaluated with a highly specific model such as SM3, which would identify and remove any misclassified unirradiated samples remaining in the set and leave only true positives (TPs). In one embodiment, the method of selection of the specific signature involves avoiding inclusion of genes, gene products or biochemical pathways, whose expression changes are shared between radiation exposed and any of the confounder populations. The method encompasses both the sequential signatures that are derived as well as the adversarial (generative) machine learning, which are detailed explained in the following sections of the description.

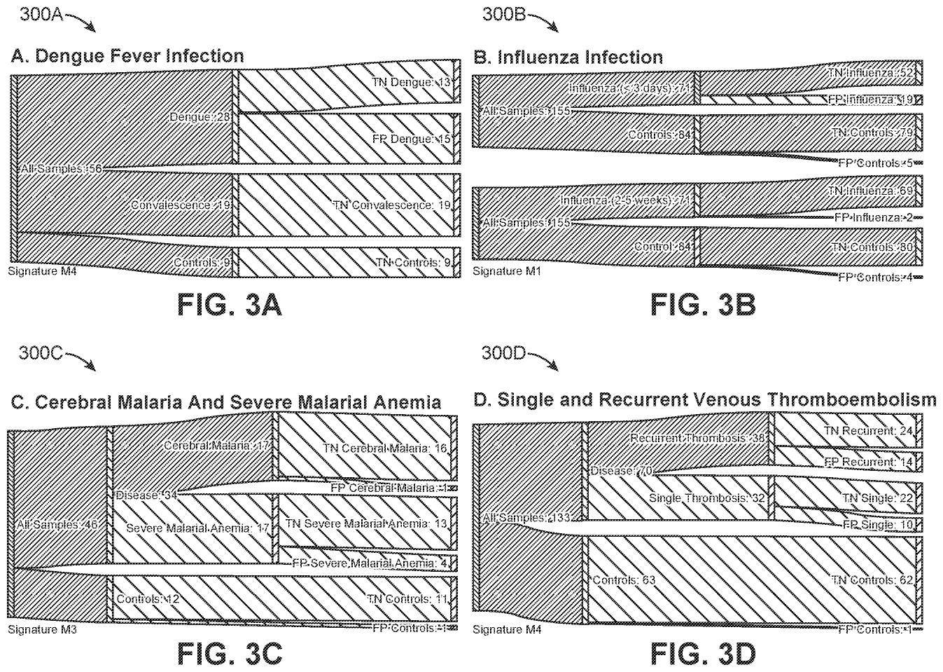

FIG. 3A—FIG. 3D illustrates graphs (300A-300D) showing the performance confounding disease samples stratified by subphenotypes using traditionally-validated radiation signatures, according to an embodiment of the present invention. The Sankey diagrams indicate those diseased patients and controls properly (TN) and improperly classified (FP) by a radiation gene signature. Referring to FIG. 3A, the radiation signature M4 incorrectly classified 53% of Dengue-infected patient as irradiated, however all convalescent patients were properly classified (0% FP). Referring to FIG. 3B, similarly, the FP rate of M1 decreased considerably after patient recovery (27% FP rate [N=19] against samples <3 days after symptoms; 3% [N=2] after 2-5 weeks). Referring to FIG. 3A, the FP rate of M3 was higher for severe malarial anemia patients versus those with cerebral malaria, suggesting that the differential expression caused by the two infection types may diverge in such a way that is measurable by M3. Referring to FIG. 3D, conversely, the FP rate of venous thromboembolism patients by M4 was not influenced on whether the disease was recurrent.

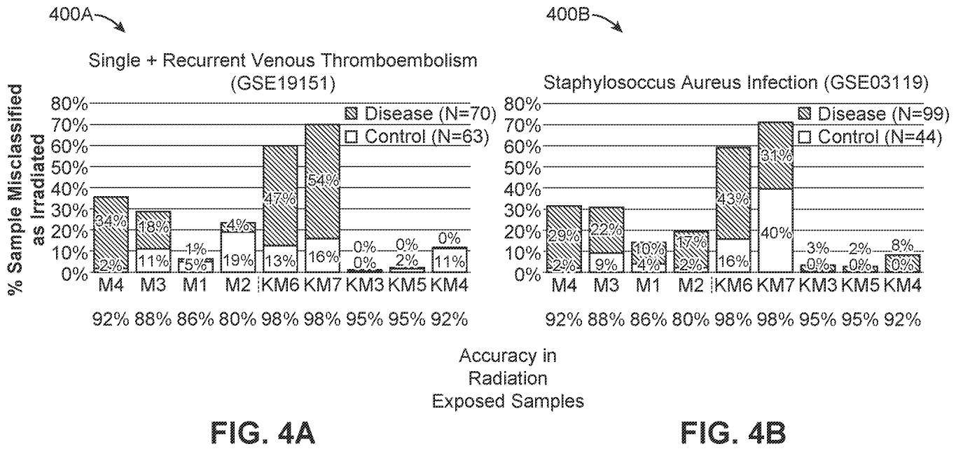

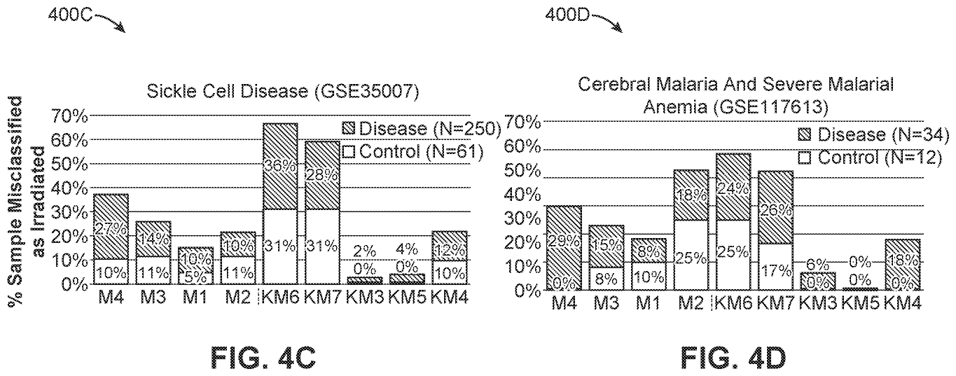

FIG. 4A-FIG. 4D are bar graphs (400A-400D) illustrating percentage of samples misclassified as irradiated, according to an embodiment of the present invention. The radiation gene signatures M1-M4 and KM3-KM7 performed well when predicting radiation exposure. However, many of these models falsely predicted individuals with blood-borne disorders (thrombosis and sickle cell disease (SCD)) and infectious diseases (S. aureus and Malaria) as irradiated (% FP provided for individuals with the indicated disease, and controls). In general, the FP rate was high for all traditional validated (M1-M4) and most k-fold validated models (KM4, KM6 and KM7). Models KM3 and KM5 had a low FP rate across all datasets tested.

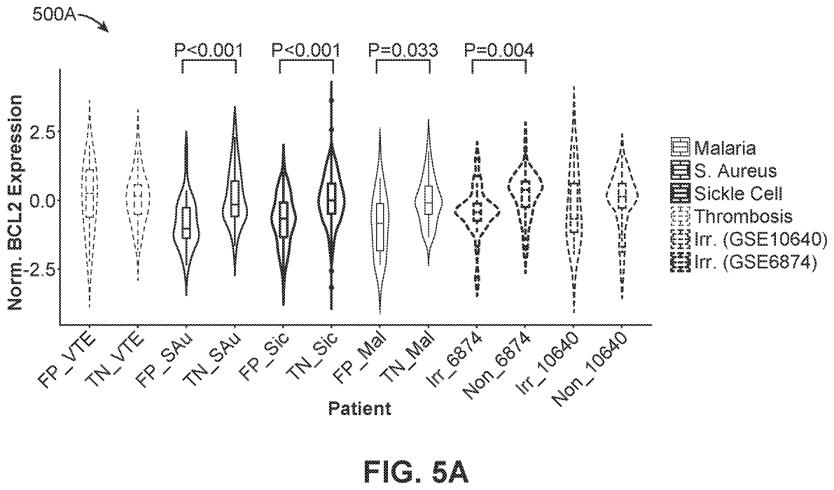

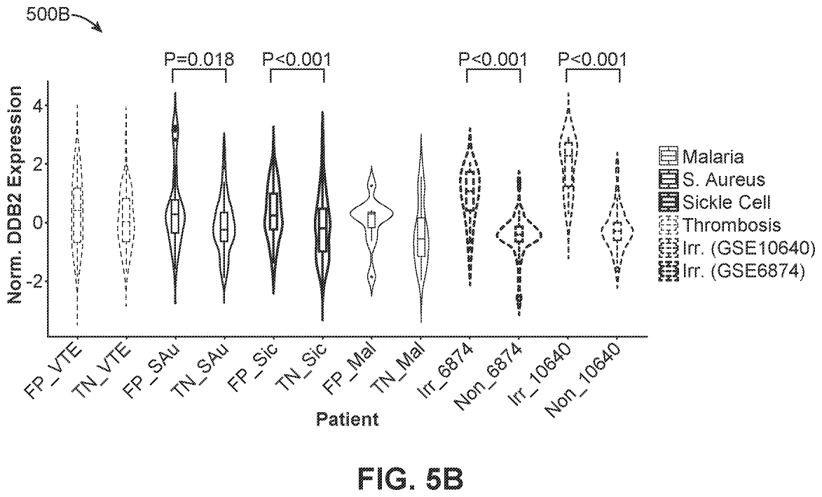

FIG. 5A-FIG. 5B are violin plots (500A-500B) illustrating DDB2 and BCL2 expression from hematological disorder and radiation-exposure datasets, according to an embodiment of the present invention. The normalized expression of various confounder datasets (VTE: Venous Thromboembolism; SAu: S. aureus; Sic: Sickle Cell; and Mal: Malaria) for the genes A) DDB2 and B) BCL2 were plotted as violin plots, where expression has been divided between diseased individuals predicted irradiated (FP) and non-irradiated (TN) by M4. Expression of these genes in irradiated (Irr.) and non-irradiated (Non) individuals are presented in light and dark red (GSE6874 and GSE10640, respectively). Significant differential expression between FP and TN (by T-Test) are indicated with square brackets.

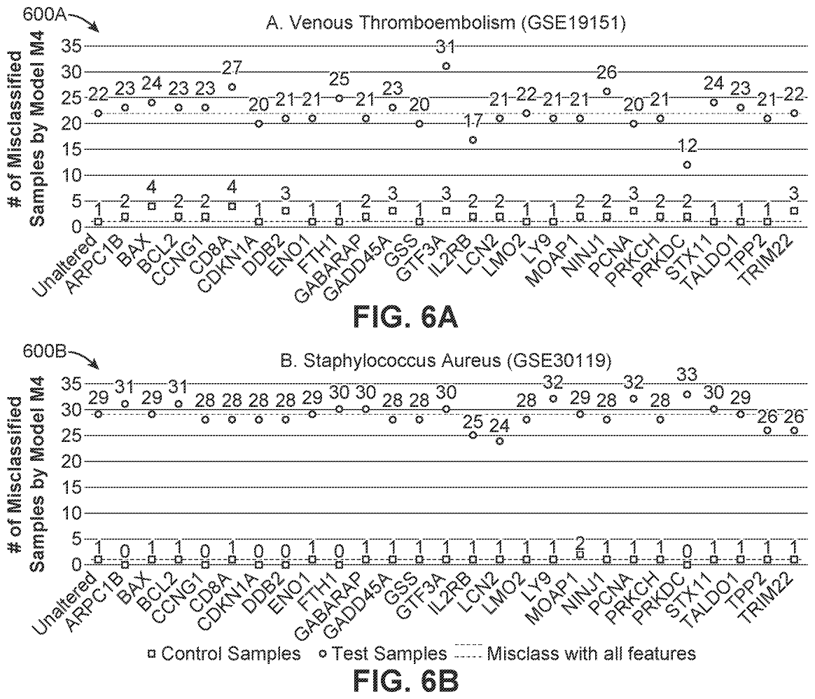

FIG. 6A-FIG. 6B illustrates graphs (600A-600B) indicating the impacts of removal of genes from signatures as applied to misclassification of confounding datasets, according to an embodiment of the present invention. The accuracy of the M4 was significantly influenced by hematological confounders such as venous thromboembolism and S. aureus infection. M4 misclassified diseased individuals (represented as circles) far more often than controls (represented as squares). Feature removal analysis of M4 determines if a particular gene was contributing to the FP rate by observing how accuracy changes when a gene is removed. While M4 accuracy improved with the removal of PRKDC, IL2RB and LCN2, no individual gene restored misclassification back to control levels suggesting multiple genes are confounded by these diseases.

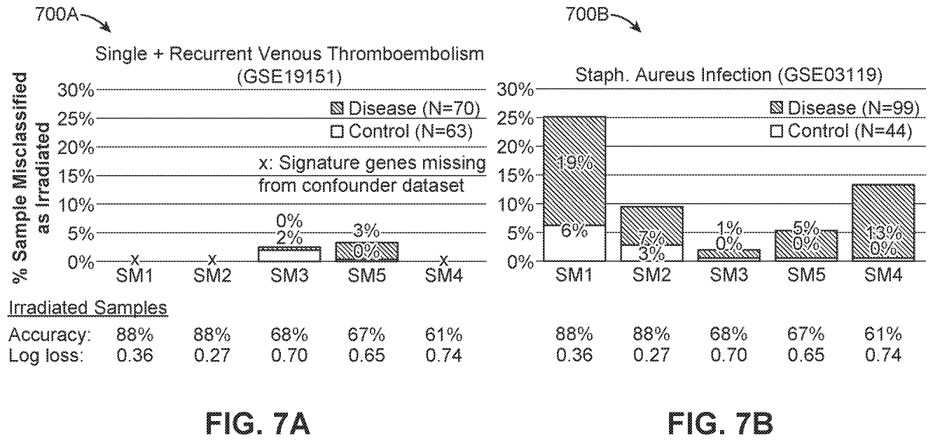

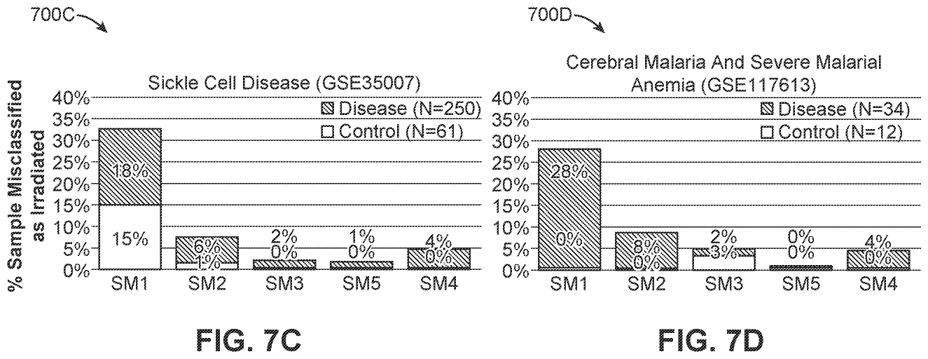

FIG. 7A-FIG. 7D illustrates bar graphs (700A-700D) of secretome-derived gene signatures that reduce misclassification of confounder phenotypes, according to an embodiment of the present invention. The confounding effect of blood-borne diseases could be mitigated by deriving signatures that lack the genes present in the confounded radiation signatures, in particular those which play a role in DNA response and apoptosis. Radiation signatures which consist exclusively of genes encoding plasma secreted proteins were derived following the same basic approach of Zhao et al (2018a). These models showed generally favorable performance when tested against an independent radiation dataset by k-fold validation. Five secretome radiation signatures were derived consisting of 7-75 genes (SM1-SM5; Table 3). Two models (SM3 and SM5) show high specificity across all hematological conditions tested.

We determined high misclassification rates of radiation gene expression signatures in unirradiated individuals with various blood borne disorders relative to controls. This was confirmed with a second set of k-fold validated radiation signatures from our previous study (Zhao et al. 2018a). The same analysis was performed on non-irradiated expression data from individuals with other hematological conditions, which extended the spectrum of other abnormalities misclassified as exposed to radiation. Some of the same genes that are induced or repressed by radiation exhibit similar changes in direction and magnitude in infections and hematological conditions (for example, DDB2, BCL2). Signatures derived from more recent microarray platforms that contain key radiation response genes missing in our previous study (e.g., FDXR, AEN) were also prone to misclassifying hematological confounders as false positives. By assessing the performance of each model and rejecting signatures with a high rate of false radiation diagnoses in confounding conditions or phenotypes, many individuals with these comorbidities might be ineligible for these radiation gene signature assays.

Given the similarities between the changes in genes expressed by radiation and the confounding hematological disorders, this raises the question as to whether therapeutic radiation in individuals with these conditions would be contraindicated, since they might exacerbate, increase severity, or compromise treatment outcomes. Indeed, there is already some evidence of interaction of these comorbidities with radiation exposure. A side effect of therapeutic radiation, radiodermatitis, is associated with S. aureus infections (Hill et al. 2004). In a large meta-analysis, radiation therapy was contraindicated for individuals with venous thrombosis (Guy et al. 2017).

The symptoms of prodromal influenza and Acute Radiation Syndrome (ARS) significantly overlap. During influenza outbreaks, this could impact accurate and timely diagnosis of ARS. Expression-based bioassays might not improve this diagnostic accuracy, since traditional radiation signatures maximize sensitivity without accounting for the diminished specificity due to underlying hematological conditions. Other highly specific tests for radiation exposure, such as the dicentric chromosome assay, require longer to perform, but analysis times are now as fast or faster than commercial gene expression assays, less variable, and can be more accurate (Rogan et al. 2016; Liu et al. 2017; Shirley et al. 2017; Li et al. 2019, Shirley et al. 2020). Existing gene expression assays will need to address the false positive results obtained for individuals with hematopoietic confounding conditions before they can be used in general populations, who may not have a history of these conditions or who may have been pre-screened as a precondition to military or space deployment.

Use of matched, unirradiated controls provides a measure of sensitivity and dynamic range of the derived radiation gene signature. Given that responses to different hematopoietic pathologies by leukocytes share common gene elements, the specificity of signature for radiation exposure would, under ideal circumstances, be expected to exclude detection of other pathologies. Negative controls are typically people who are currently without disease or mild symptoms. In a nuclear incident or accident, the exposed population will include many others with underlying comorbidities. Application of radiation signatures derived by maximizing sensitivity in this population could lead to inappropriate diagnosis, and possibly treatment for ARS. We derive an assay design, based on sequentially-applied ML signatures, that should improve the specificity of radiation gene expression assays in these individuals, and across the general population.

The cumulative incidences of these confounders are not rare, especially influenza which affected approximately 11% of the US population during the 2019-2020 flu season (11,575 per 100,000; https://www.cdc.gov/flu/about/burden). Frequency of dengue fever was also high in the Caribbean (2,510 per 100,000), Southeast Asia (2,940 per 100,000) and in South Asia (3,546 per 100,000; based on cases from 2017 [Zeng et al. 2017]). The annual prevalence of S. aureus bacteremia in the US is 38.2 to 45.7 per 100,000 person-years (El Atrouni et al. 2009; Rhee et al. 2015), but is higher among specific populations, such as hemodialysis patients. There are between 350,000 and 600,000 cases (200 per 100,000) of deep vein thrombosis and pulmonary embolism occur in the US every year (Anderson et al. 1991). Furthermore, there are over 100,000 individuals with SCD in the US (33.3 per 100,000; Hassell, 2010). Malaria is also common in sub-Saharan Africa in 2018 (21,910 per 100,000; World Health Organization, 2018). The prevalence of these diseases makes it clear that they could very well have a severe impact on assessment in a population-scale radiation exposure event.

Exploring the basis of these confounding disorders or phenotypes could facilitate strategies that mitigate against FP radiation exposure assignments. Riboviral infections have been proposed to sequester host RNA binding proteins, leading to R-loop formation, DNA damage responses, and apoptosis (Rogan et al. 2021). We propose that expression of some key radiation signature genes appear to be affected by such infections. Neutrophil extracellular traps (or NETs) may be another common link that explains these FP predictions to radiation exposure (Qi et al. 2020). An early step in the formation of these structures is chromosome decondensation followed by the fragmentation of DNA which act as extracellular fibers which bind pathogens (such as S. aureus) in a process similar to autophagy in neutrophils (NETosis). This process would likely activate DNA damage in neutrophils, and some of the same DNA damage response genes that are activated (DDB2, PCNA, GADD45A) and repressed (BCL2) after radiation exposure are also similarly regulated after infections such as S. aureus. NETosis also contributes to the pathogenesis of numerous non-infectious diseases such as thrombosis (Demers and Wagner, 2014; Collison, 2019) and SCD (Hounkpe et al. 2020), in addition to autoimmune disease (He et al. 2018) and general inflammation (Delgado-Rizo et al. 2017). If the origin of the FPs is confined to this lineage, then a comparison of the predictions of our traditionally validated signatures using data from the granulocyte versus lymphocyte lineages in individuals with these conditions should reveal whether NETosis is the likely etiology of the confounder expression phenotypes, or possibly even in radiation treated cells. To do this for radiation exposed cells would require RNASeq data from these isolated cell populations (Ostheim et al. 2021). We would expect FPs in the confounder populations using signatures derived from myeloid-derived (rather lymphoid-derived) lineages.

Confounding conditions will affect the precision of other assays and biomarkers that are routinely used to assess radiation exposure. Indeed, some of these are well known in the published literature. For example, elevated levels of γ-H2AX occurs in melanoma (Warters et al. 2005), cervical cancer (Banath et al. 2004; Yu et al. 2006), colon carcinoma, fibrosarcoma, glioma, osteosarcoma, and neuroblastoma (Sedelnikova and Bonner 2006). The DNA damage detected by this marker is characteristic of the development of cancer (Banath et al. 2004; Warters et al. 2005; Sedelnikova and Bonner 2006; Yu et al. 2006). Colonocytes from patients with ulcerative colitis also have elevated γ-H2AX (Risques et al. 2008). Increased expression of γ-H2AX is such a reliable biomarker in this context, that it has been suggested for early cancer screening and cancer therapy monitoring (Sedelnikova and Bonner 2006), which would make its use to assess radiation exposure problematic in such patients. Aside from its application in radiation damage assessment, the cytokinesis block micronucleus assay (Fenech 2010) is actually a multi-target endpoint for genotoxic stress from exogenous chemical agents (Kirsch-Volders et al. 2011; Fenech et al. 2016, Kirsch-Volders et al. 2018) and deficiency of micronutrients required for DNA synthesis and/or repair (folate, zinc; Beetstra et al. 2005; Sharif et al. 2012). Zinc depletion/restriction also increased γ-H2AX (Mah et al. 2010) suggesting increased DNA breakage, which has been confirmed by the comet assay (Song et al. 2009).

Many radiation response genes were frequently selected as features for multiple signatures, and includes genes with roles in DNA damage response (CDKN1A, DDB2, GADD45A, LIG1, PCNA), apoptosis (AEN, CCNG1, LY9, PPM1D, TNFRSF10B), metabolism (FDXR), cell proliferation (PTP4A1) and the immune system (LY9 and TRIM22). In general, the removal of these genes did not significantly alter the FP rate against confounder data. However, the removal of LIG1, PCNA, PPMID, PTP4A1, TNFRSF10B, and TRIM22 could partially decrease misclassification of influenza samples in some models, as well as DDB2 for dengue (in addition to S. aureus and Polycythemia Vera). Many of these genes in our models are also present in other published radiation gene signatures and assays (Paul and Amundson, 2008; Lu et al. 2014; Oh et al. 2014; Port et al. 2017; Tichy et al. 2018; Jacobs et al. 2020). Paul and Amundsen (2008) developed a 74-gene radiation signature that comprises of 16 genes present in the human models (and an additional 3 exclusively in mouse models) reported in Zhao et al. (2018a), including CDKNIA, DDB2 and PCNA (AEN and FDXR are also present). Similarly, three of the 5 biomarkers implicated in Tichy et al. (2018) were also commonly selected (CCNG1, CDKNIA, and GADD45A), as were 5 of the 13 genes in the radiation assay described in Jacobs et al. (BAX, CDKNIA, DDB2, MYC and PCNA). While we cannot determine the impact on the accuracy of their signatures for confounders, it is evident is that some genes that are included in these and other gene signatures (such as DDB2) can have a profound impact on the misclassification of individuals with confounding conditions.

A sequential approach in which ML predictors that are highly sensitive to radiation exposures (but affected by confounders) are used in combination with high-specificity signatures could be used to unequivocally identify true positive exposures, as shown in FIG. 7A-FIG. 7D. First, a sample would be assessed with a highly sensitive radiation signature, such as M4. All predicted positive samples would then be re-evaluated with a high specificity signature which identifies and removes samples misclassified as irradiated, resulting in a higher performance assay that predominantly or exclusively labels truly irradiated samples. Besides radiation exposure, the application of successive gene signatures, each optimized respectively to maximize sensitivity and specificity, is a general strategy for improving accuracy of molecular diagnoses for a wide spectrum of disease pathologies.

The same predicted positive sample set could alternatively be evaluated by multiple gene signatures specific for various confounding variables (e.g., a model for influenza infection, for thrombosis, and other conditions). Another alternative method (that would not require separate signatures to maximize sensitivity and specificity) would involve training and validation of adversarial networks during model derivation ML steps, where radiation positive samples are contrasted with one or more confounding datasets, in particular those samples determined to be FP using the currently used algorithm (Goodfellow et al. 2014). This would create ML signatures that includes radiation response genes that are not affected by the tested confounding condition(s). Ensuring that both the positive test and negative control samples in training sets properly account for the frequencies of confounding conditions in the population may also offer an unbiased solution to the issue of confounders.

### EXAMPLES

The experiments and results of operation to which this invention applies will be described as follows.

### Example-1

Evaluating Specificity of Radiation Gene Signatures with Expression of Genes in Confounding Hematological Conditions

Radiation gene signatures derived by biochemically-inspired machine learning in this study and in Zhao et al. (2018a) were used to evaluate publicly-available patient datasets of individuals and controls for hematological disorders using traditional validation methods (‘regularValidation_multiclassSVM.m’ from Zhao et al. [2018b]). Performance was evaluated by observing how often non-irradiated individuals were misclassified as radiation exposed by these models. The confounding effect of the various blood-borne disorders tested was measured by comparing divergence between the FP (false positive) rates in patient vs. control samples. We explored the degree to which specific genes from each signature contributed to misclassification by performing feature removal analysis (Mucaki et al. 2019), where genes within a signature are individually removed from the model which is then reassessed against the test (confounder) datasets. Known radiation responsive genes in the confounder datasets of correctly vs. misclassified samples were visualized using violin plots of gene expression. These display weighted distributions the differential gene expression from each confounder datasets which were properly and improperly classified as irradiated by the radiation gene signatures (created in R language [i386 v4.0.3] using the library ggplot2). Misclassification of confounder sub-phenotypes was stratified using Sankey diagrams. This analysis delineates FP and true negative predictions (at the individual level) of groups of diseased patients and controls from these datasets according to predictions of the designated, specific radiation gene signature.

### Example—2

### Initial Evaluation of Candidate Genes in Radiation Gene Expression Datasets for Machine Learning

We derived new gene expression signatures by leave-one-out and K-fold cross-validation from microarray data based on more recent comprehensive gene datasets (GEO: GSE85570 and GSE102971) besides those we previously reported (Zhao et al. 2018a). Only some of the 1,011 curated genes were present on these microarray platforms, including 864 genes in GSE85570 and 971 genes of GSE102971. After normalization, gene rankings by mRMR between GSE102971 and GSE85570 were similar. In GSE85570, FDXR were ranked first, while AEN was top ranked in GSE102971 (FDXR was ranked 38th). DDB2 was top ranked in GSE6874 and GSE10640 (Zhao et al. 2018a), both of which lacked FDXR and AEN. Radiation-response genes among the top 50 ranked present in all 4 datasets included BAX, CCNG1, CDKN1A, DDB2, GADD45A, PPM1D and TRIM22.

ERCC1 was chosen as the second-ranked gene in GSE102971, even though its MI was 31-fold lower than the top ranked gene, AEN. MI of the second-ranked genes in GSE6874 (RAD17) was 7-fold lower than the first (DDB2), while GSE10640 (CD8A) showed a 4-fold difference. Six of the top 50 genes in GSE102971 exhibited <10% of the MI of AEN (3 genes for GSE6874; none of the top 50 in GSE10640 and GSE88570 were <10% of the top ranked gene). Genes with low MI values are likely to make little or no contributions to predictions by gene signatures and introduce noise into ML models. Selection of low MI genes by ML feature selection likely reduces accuracy of gene signatures during validation steps. In the future, signature derivation will set a minimum MI threshold for ranking genes by mRMR.

The overall levels of MI for top ranked genes in GSE85570 (0.72 bits for AEN) and GSE102971 (0.82 bits for FDXR) were comparable. In GSE102971, the genes with the highest MI were AEN, DDB2, FDXR, PCNA and TNFRSF10B (closely followed by BAX). While each were found in the top 50 ranked genes, some rankings were decreased to minimize redundant information (FDXR and AEN are ranked #38 and #41 in the GSE102971 dataset, respectively). MI for the top ranked genes in GSE6874 and GSE10640 were lower by comparison (0.31 and 0.47 bits for DDB2, respectively); the depressed maximum MI values in these datasets may, in part, be related to reduced numbers of eligible genes on these microarray platforms.

TABLE 3
Radiation Gene Signatures including only Secreted
Genes derived from GSE6874 and GSE10640 Datasets

| | | Validation | |
|---|---|---|---|
| | | Misclassification | |
| | FS. | K- | |
| Signature | Algorithm | Fold ¹ | Traditional |

a) Derived from Radiation Dataset GSE6874[GPL4782] and Validated on
GSE10640[GPL6524]

| | | | | |
|---|---|---|---|---|
| SM1 | PDE7A FBXW7 CLCF1 ALB IDUA USP3 SLPI | CS | 0.12 | 0.25 |
| | COASY MFAP4 LTBP1 VPS37B VEGFA IRAK3 | FS | | |
| SM2 | PDE7A FBXW7 CLCF1 ALB IDUA USP3 SLPI | BS | 0.12 | 0.27 |
| | COASY MFAP4 LTBP1 VPS37B VEGFA IRAK3 | FS | | |
| | MZB1 DHH GRN AEBP1 CNPY3 NUCB1 RDH11 | | | |
| | CXCL3 POFUT1 CST1 ARCN1 PLA2G12A ERAP2 | | | |
| | GOLM1 B3GAT3 ADAMTS9 FKBP9 ALDH9A1 LY86 | | | |
| | HARS2 PRSS21 RETN C1GALT1 MGAT2 FUCA1 | | | |
| | TTC19 MANF LUM GALNT15 APOM NME1 ATMIN | | | |
| | GPX4 POLL LY6H SMARCA2 | | | |

b) Derived from Radiation Dataset GSE10640[GPL6522] and Validated on
GSE6874[GPL4783]

| | | | | |
|---|---|---|---|---|
| SM3 | TRIM24 TOR1A GRN HP RBP4 PFN1 FN1 | FS | 0.32 | 0.49 |
| | | FS | | |
| SM4 | XGL1 CDC40 PTGS2 DHX8 NENF PTX3 WNT1 | CS | 0.39 | 0.32 |
| | CTSW TINF2 AOAH VPS51 TOR1A HINT2 CRTAP | FS | | |
| | SUCLG1 TF EDEM2 LAMA5 AGPS TFPI WFDC2 | | | |
| | SRGN SIL1 PPOX AMY2A NUBPL GARS LRPAP1 | | | |
| | VPS37B PNP C3orf58 HP SPOCK2 NME1 GRN | | | |
| | TRIM24 MRPL34 SRP14 THOC3 RNASE6 RBP4 | | | |
| | MSRB2 RNASET2 TGFBI PRDX4 GLA GLB1 PFN1 | | | |
| | GDF15 VCAN TRIM28 TAGLN2 TIMP1 IPO9 CPVL | | | |
| | MANBA CEP57 RNF146 PF4 RETN HCCS DPP7 | | | |
| | RNASE2 QPCT AHSG CTSC LYZ B2M EMILIN2 | | | |
| | STOML2 LCN2 | | | |
| SM5 | TRIM24 IRAK3 PPP1CA MTX2 FBXW7 PFN1 | FS | 0.33 | 0.38 |
| | SDHB CTSC MSRB2 | FS² | | |

Additional Metrics for these signatures can be found in Suppl. Table S6A;
¹ Tested using K-Fold validation methods (where K = 5);
²Derived from the top 50 genes by ranked mRMR (Suppl. Table S1);
FS—Feature Selection metrics

### Example—3

### Radiation Gene Signature Performance in Blood-Borne Diseases

The specificity of previously-derived radiation signatures selected after K-Fold validation (KM1-KM7) and traditional validation (M1-M4; Zhao et al. 2018a) was assessed with normalized expression data of patients with unrelated hematological conditions rather than evaluating unirradiated healthy controls. Signatures M1 and M2 (from GSE10640) and M3 and M4 (from GSE6874) were assessed with expression datasets of Influenza A (GSE29385, GSE82050, GSE50628, GSE61821, GSE27131) and Dengue fever (GSE97861, GSE97862, GSE51808, GSE58278) blood infections (Rogan et al. 2021). FPs for radiation exposure were defined as instances where the misclassification rates of individuals with the disease diagnosis exceeded normal controls. A clear bias towards FP predictions of infected samples relative to controls was evident with all of these radiation gene signatures (Rogan et al. 2020; extended data). Dissection of the ML features responsible implicated 10 genes contributing to misclassification, including BCL2, DDB2 and PCNA. We determine whether other conditions confound the accuracy of additional human gene signatures (KM1-KM7; Zhao et al. 2018a) as well as newly derived signatures from more recent radiation gene expression datasets.

FP misclassification of viral infections were also evident with KM1-KM7 (Zhao et al. 2018a). KM6 and KM7 (derived from GSE6874) misclassify all Influenza and most Dengue fever (GSE97861, GSE51808 and GSE58278) datasets of patients at higher rates than uninfected controls. KM3-KM5 exhibited low FP rates in influenza relative to other models, but Dengue viral GSE97862, GSE51808 and GSE58278 exhibited higher FP rates in infected samples vs uninfected controls. Interestingly, KM5 is the only high sensitivity human gene signature in which DDB2 is not present; this gene was previously shown to contribute to high FP rate (Rogan et al. 2021). Among KM3, KM4 and KM5, KM5 is the preferred signature, exhibiting the highest sensitivity and specificity of an individual signature for detection of radiation exposure. KM1 and KM2, which were derived from a third radiation dataset (GSE1725), often misclassified in virus infected samples relative to controls (KM1 only: GSE97861; KM2 only: GSE82050, GSE27131, GSE97862 and GSE50628; both KM1 and KM2: GSE51808, GSE58278, and GSE61821). In some datasets, these models also demonstrated high FP rates in controls.

Expression changes in signature genes resulting from influenza A and Dengue fever infections are stabilized back to control levels after either convalescence or at the terminal stage of infection. For example, M4 exhibited a 54% FP rate for samples from dengue-infected individuals 2-9 days after onset of symptoms (GSE51808). All samples obtained >4 weeks after initial diagnosis, however, were correctly classified as non-irradiated by this signature (as shown in FIG. 3A). The influenza gene expression dataset GSE29385 longitudinally sampled infected patients after initial symptoms at <72 hours [Ti], 3-7 days [T2], and 2-5 weeks [T3]. FPs were significantly decreased at the last time point using nearly all models (19 infected samples misclassified by M1 at T1 was reduced to 2 cases at T3; FIG. 3B). This result highlights both the impact that influenza-induced transcriptional changes have on model accuracy and the reversion of these changes after convalescence or late-stage disease.

### Example—4

### Specificity of Radiation Signatures Using Datasets of Other Hematological Conditions.

Radiation signatures that distinguish blood gene expression due to ionizing radiation at different rates, levels, and types of energy also need to differentiate changes that result from other hematological conditions. We investigated whether radiation gene signature accuracy was compromised by the presence of other blood borne infections and non-infectious, non-malignant hematological pathologies with publicly-available expression data from blood-borne disorders with adequate sample sets (>10 individuals with corresponding control samples). Initially, GEO datasets from patients with single and recurrent venous thromboembolism (GSE19151), community acquired S. aureus infection in vivo (GSE30119), cerebral Malaria and severe Malarial anemia (GSE117613), pediatric SCD (GSE35007), idiopathic portal hypertension (GSE69601), polycythemia vera (GSE47018) and aplastic anemia (GSE16334) were considered. The idiopathic portal hypertension dataset was excluded due to insufficient sample numbers. We then determined recall levels for signatures M1-M4 and KM3-KM7 evaluated with these datasets, under the assumption that these models were expected to predict all individuals as non-irradiated.

S. aureus infections were misclassified as FPs by all signatures except KM7, as shown FIG. 4A-FIG. 4B. Each radiation gene signature was observed to be confounded by some but not all blood-borne disorders and infections. High FP rates were observed for M1 and KM5—SCD and S. aureus; M2—S. aureus; M3 and M4—malaria, SCD, venous thrombosis, polycythemia vera and S. aureus; KM3 and KM4—malaria, SCD and S. aureus; KM6—venous thrombosis, polycythemia vera, S. aureus; KM7—Malaria, venous thrombosis and polycythemia vera. The malaria dataset stratified patients with either cerebral malaria (CM) and severe malarial anemia (SMA) (GSE117613; Nallandhighal et al. 2019). The SMA subset contains the majority of the FPs, as shown in FIG. 3C. The venous thrombosis dataset (GSE19151; Lewis et al. 2011), which was stratified as either single vs recurrent venous thromboembolism, exhibited similar FP rates for both subsets, as shown in FIG. 3D. Predictions of radiation exposure by M3, M4, KM6 and KM7 are confounded by transcriptional changes resulting from different blood-borne conditions, while M2 and KM5 are the least influenced by these conditions. Nevertheless, both KM2 and KM5 were confounded by S. aureus infections. Aplastic anemia did not increase FP rates compared to controls for any of the signatures, consistent with our previous findings (Rogan et al. 2021).

Signatures with high sensitivity for radiation, M4, KM6 and KM7, are confounded by either the viral or blood-borne infections and other non-infectious blood disorders. The genes within these signatures with the most profound impact on accuracy of these signatures were determined by evaluating differential expression of these genes in samples that were correctly (TN) vs incorrectly (FP) classified. The normalized gene expression distributions of TN and FP samples using these radiation signatures in Malaria, S. aureus, SCD and Thrombosis (also Influenza A and Dengue fever) were visualized as violin plots (Mucaki et al. 2021). Expression of BCL2 for the S. aureus, SCD and malaria samples was significantly lower in FP samples relative to TNs with M4 (p<0.05 with Student's t-test, assuming two-tailed distribution and equal variance), similar to the effect of radiation exposure on expression of this gene, as shown in FIG. 5A. These same FP individuals have significantly higher DDB2 expression in both S. aureus and SCD, as shown in FIG. 5B. Increased DDB2 expression was also observed for FPs using KM6 and KM7. For both the BCL2 and DDB2 genes, differences in expression in samples classified as TN and FP were congruent with changes from radiation exposure. Genes that may contribute to misclassification include GADD45A in M4 (higher expression in diseased individuals vs. controls and induced by radiation exposure), and PRKCH and PRKDC, respectively, in KM6 and KM7 (decreased expression in FPs and in response to radiation). BAX, which is induced by radiation, is similarly expressed in FP and TN samples, and probably does not contribute to misclassification by M4.

To determine the extent to which each gene contributes to the FP rates in each signature, gene features were removed individually, the signature was rebuilt and misclassification rates were reassessed for each confounding condition (Supplementary Table S3A [M1-M4] and S3B [KM3-KM7]). Removing genes individually from gene signatures M1, M3, M4, KM3, KM5 and KM7 does not significantly alter the previously observed misclassification rates for the blood disorders. The FP rates of M4 for thrombosis samples were most impacted by elimination of PRKDC (DNA double stranded break repair and recombination) and IL2RB (innate immunity/inflammation), respectively improving accuracies by 10% and 5%, as shown FIG. 6A, which was still significantly above the FP rates observed in controls. However, the removal of these genes does not improve the FP rate of M4 in S. aureus infected samples, as shown in FIG. 6B. Thus, no single gene feature dominated the predictions of these signatures and could account for the misclassified samples. Removal of DDB2, GTF3A or HSPD1 from KM4 significantly decreased its FP rate to the malaria dataset (18% to 0-3%). Similarly, removal of DDB2 from M2 and KM6 lead to the complete elimination of positives in both patients and controls. However, the removal of DDB2 from these models was previously shown to severely reduce the true positive rate against irradiated samples (Zhao et al. 2018a), thus these genes cannot be eliminated without affecting the sensitivity of these signatures to accurate identify radiation exposures.

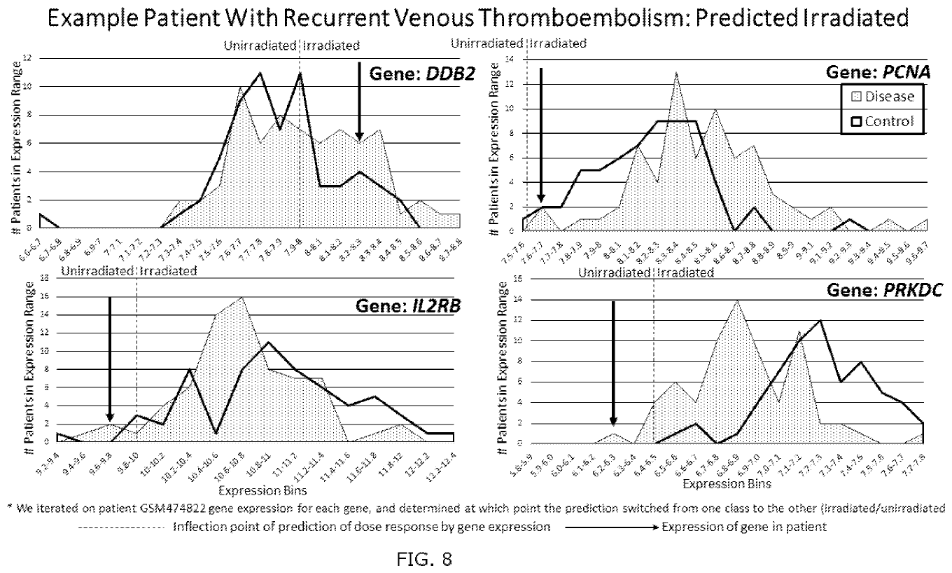

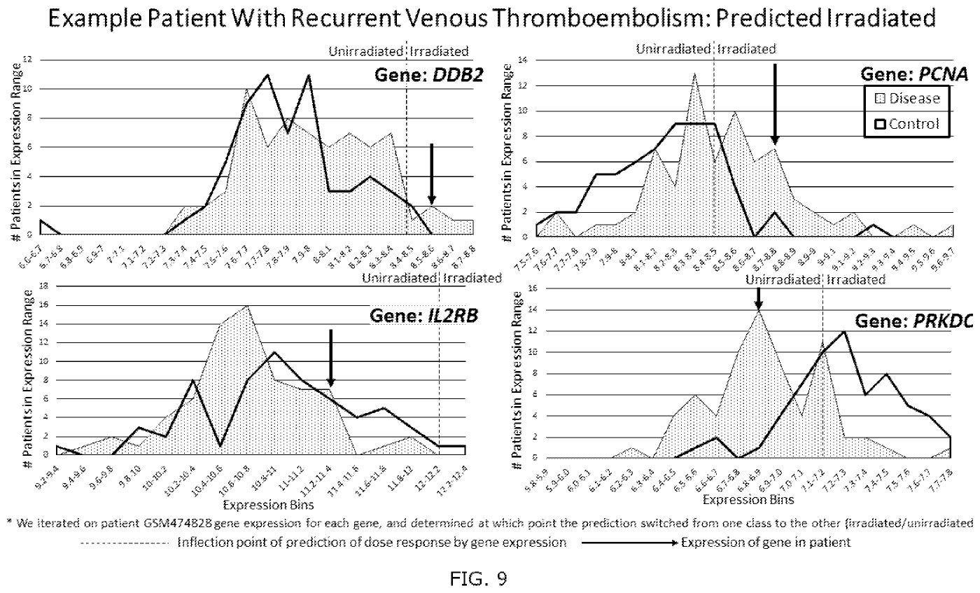

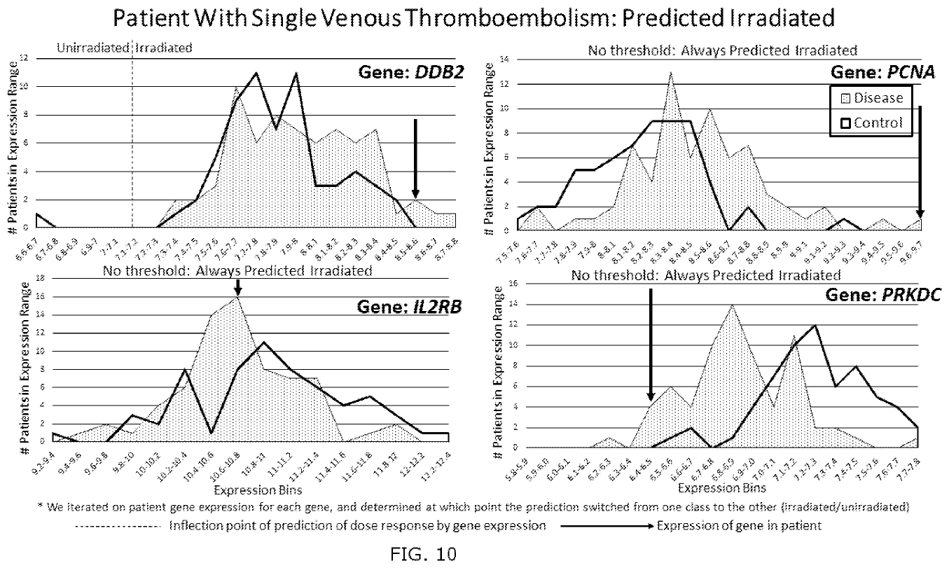

The impact of expression levels of individual genes comprising signatures can also be evaluated by threshold mapping, which computationally modifies the expression levels of a gene in a dataset to determine the expression level required to change the predicted outcome of the ML model (i.e., the inflection point of the prediction that distinguishes exposed from non-irradiated samples). The threshold is visualized in the context the actual expression value in an individual relative to a histogram of expression of the entire population in the validation dataset. Actual expression values that are close to this threshold can indicate the reliability of either the radiation exposure prediction or of misclassification by the model due to an underlying confounding condition. Expression levels and thresholds of DDB2, IL2RB, PCNA and PRKDC for 3 individuals with thromboembolism (GSE19151) predicted to be irradiated by M4 (GSM474819, GSM474822, and GSM474828) are indicated in FIGS. 8-10. Reduction of DDB2 expression corrected misclassification for all three patients, as did decreasing PCNA expression in GSM474822 and GSM474828. Increasing IL2RB and PRKDC expression of these two patients corrected their FP classification. These results correspond to the effects of radiation on the expression of these genes in the GSE6874 and GSE10640 datasets, e.g., induction of DDB2 and PCNA, repression of IL2RB and PRKDC (Mucaki et al. 2021). The expression changes of DDB2, PCNA and PRKDC in these patients were small relative to the dynamic range across the entire dataset, but were sufficient to alter predictions of the signatures. Conversely, changes in expression of PCNA, IL2RB nor PRKDC were unable to modify the prediction of M4 in GSM474819. Only a large decrease in DDB2 expression to levels below nearly the entire population of thromboembolism patients was able to alter the classification of this individual. This illustrates that DDB2 expression has a strong impact in model prediction accuracy (as previously noted; Zhao et al. 2018a). Nevertheless, the combined expression of most of the genes which constitute the signature ultimately determine the outcome of the classifier. Incorrect classifications where expression values are close to the model's predictive inflection point should be considered when assessing misclassification accuracy.

### Example—5

Misclassification of Confounders with Radiation Gene Signatures Derived from Alternate Microarray Platforms

FSFS- and BSFS-derived radiation gene signatures were derived from the top 50 mRMR ranked genes of the GSE85570 and GSE102791 expression datasets using varying combinations of the C and a parameters, minimizing on sample misclassification. Genes selected in GSE85570-based signatures included BAX, FDXR, XPC, DDB2 and TRIM32. All signatures from this dataset exhibited low misclassification rates (<5 by cross-validation. GSE102791 contained sets of 20 samples, each irradiated at different absorbed energy levels (0 vs 2, 5, 6, and 7 Gy). Different ML models were derived either utilizing the full dataset or based a combination of 2 and 5 Gy samples. The models derived from either subset of GSE102791 also exhibited very low misclassification rates by cross-validation (0-1 samples) or by log-loss (<0.01). Common genes selected from signatures derived from GSE102791 include AEN, BAX, TNFRSF10B, RPS27L, ZMAT3 and BCL2.

The radiation gene signatures with the lowest misclassification rates from these datasets were evaluated against the blood-borne disease confounder datasets that compromised the accuracies of the M1-M4 and KM3-KM7 signatures (Zhao et al. 2018a). Misclassification rates were estimated using datasets containing the largest numbers of samples including venous thromboembolism, S. aureus infection, SCD and cerebral malaria and severe malarial anemia. The gene signature designated M5 (consisting of AEN and BCL2) showed an elevated FP rate over controls in blood samples from individuals with venous thrombosis (18%), S. aureus infection (7%), and malaria infection (33%). Misclassification of M5 was increased by 6% in SCD; a second GSE102791-derived signature containing AEN (M8) also exhibited a higher FP rate in SCD (29%). Removal of genes from M8 significantly increased the FP rate for both controls and diseased individuals, which is a limitation of models based on small numbers of genes. M9 includes BAX and FDXR and exhibited increased FP rates in thrombosis relative to controls (34-38% increased FP). Interestingly, M13 shows increased FPs in thrombosis (similar to M1-M4) while M11 does not, despite both signatures containing DDB2. Removing any of the genes from these models did not substantially alter misclassification, except for a large decrease in FP upon removal of RPS27L from M9. Both M11 and M13 exhibited high FPs in Malaria samples. BSFS models derived from GSE85570 contained FDXR, BAX and DDB2, and showed high FP in S. aureus, SCD and Malaria samples. These confounders adversely affect the accuracy of gene signatures containing radiation response genes (such as FDXR and AEN) present in both these and other recently derived signatures in the published literature.

### Example—6

### Mitigating Reduced Specificity Due to Confounding Blood-Borne Disorders Using Alternative Gene Signatures

The reduced classification accuracies of signatures by confounders are likely the result of changes to DNA damage and apoptotic gene expression in these conditions that are shared with radiation responses. ML signatures avoiding DNA damage or apoptotic genes in the presence of such confounders are hypothesized to be less prone to misclassification. Extracellular blood plasma proteins responsive to radiation exposure are generally unrelated to DNA damage response or apoptosis pathways, and represented viable candidates for derivation of alternative gene signatures that would not contain gene features present in our previous radiation signatures. For example, FLT3 ligand (FLT3LG) and alpha amylase (AMY; AMY1A, AMY2A) have previously been established as indicators for radiation exposure in blood serum (Tapio, 2013). AMY levels assess parotid gland damage (Barrett et al. 1982) and FLT3 ligand levels indicate bone marrow effects (Bertho et al. 2001). To derive a list of secreted proteins, we cross-referenced gene lists from the Human Protein Atlas Secretome database and the Plasma Protein Database (N=1,377 genes shared). Genes encoding these proteins that were not expressed in leukocytes or transformed lymphoblasts (TPM 1 in GTeX; http://gtexportal.org) were excluded. The remaining genes (N=682) were used in derivation of new radiation gene signatures using our previously described methods (Zhao et al. 2018a). One criteria that is hereby defined is that reduced specificity is evident when the unirradiated samples with confounding diagnoses are misclassified more frequently than control samples without these diagnoses with the first signature.

GM2A was the gene with the highest MI with radiation in GSE6874 (MI=0.31), while TRIM24 was highest in GSE10640 (MI=0.27). Surprisingly, MI of TRIM24 was low in GSE6874 (MI=0.05) was ranked second to last by mRMR and was not differentially expressed (p-value >0.05 by t-test). GM2A is absent from the GSE10640 dataset. Other top 50 ranked genes by mRMR in both datasets include ACYP1, B4GALT5, FBXW7, IRAK3, MSRB2, NBL1, PRF1, SPOCK2, and TOR1A.

We derived 5 independent plasma protein-encoded radiation gene signatures (‘secretome’ models) that showed the lowest cross-validation misclassification accuracy or log-loss by various feature selection strategies (labeled SM1-SM5 [Secretome Model 1-5; Table 3]). This procedure identifies signature genes that encode secreted proteins which were different from composition of largely cellular gene products found in signatures with high sensitivity to radiation exposure (eg. M1-M13, and KM1- KM7). This distinction is not limited to secretome-derived signatures. That is, other methods and strategies for deriving signatures with properties of high sensitivity and specificity, respectively, do not depend on the exclusivity of the compartmentalization of radiation-responsive genes and/or the gene products they encode. Furthermore, SM5 feature selection was limited to top 50 genes ranked by mRMR. This pre-selection step was not applied when deriving SM2 and SM3, whereas SM1 and SM4 were obtained by CSFS feature selection which selects genes sequentially by mRMR rank order without applying a threshold. SM1 and SM2 were derived from GSE6874, while SM3, SM4 and SM5 were trained on the GSE10640 radiation dataset. Genes selected that were significantly upregulated by t-test consist of SLPI (SM1, SM2), TRIM24 (SM3, SM4, SM5), TOR1A (SM3, SM4), GLA (SM4), SIL1 (SM4), NUBPL (SM4), NME1 (SM4), IPO9 (SM4), IRAK3 (SM5), MTX2 (SM5), and FBXW7 (SM5), while those downregulated include CLCF1 (SM1, SM2), USP3 (SM1, SM2), TTC19 (SM2), PFN1 (SM3, SM4, SM5), CDC40 (SM4), SPOCK2 (SM4), CTSC (SM4), GLS (SM4), and PPP1CA (SM5). The 5 models showed 12-39% misclassification (by K-Fold validation) when validated against the alternative radiation dataset. The GSE6874 dataset is missing expression data for LCN2, ERP44, FN1, GLS, and HMCN1, genes that are present in models SM3 and/or SM4. Ignoring these genes when validating these signatures fundamentally changes overall model performance, impacting model accuracy. The performance of the derived signatures was also assessed by inclusion of FLT3 or AMY, either individually or in combination. These genes did not improve model accuracy beyond the levels of the best performing signatures that we derived.

The specificity of secretome radiation gene signatures was evaluated with expression data from non-irradiated individuals with blood-borne diseases and infections. Thrombosis could only be evaluated with SM3 and SM5 due to missing genes from the SM1, SM2 and SM4 signatures. SM3 and SM5 correctly classified nearly all samples in each dataset as non-irradiated and maintained a FP rate <5% for all datasets tested, as shown in FIG. 7A-7D. The specificity of secretome gene signatures is considered to be highly specific if the gene signatures correctly classify samples with a false positive rate <5%. SM3 and SM5 contain <10 genes, were derived from GSE10640 and share the genes TRIM24 and PFN1 (ranked #1 and #21 by mRMR). Both genes are significantly differentially expressed after radiation exposure, as is TOR1A in SM3 and IRAK3, PPP1CA, MTX2, FBXW7 and CTSC in SM5. Indeed, SM3 and SM5 have the highest fraction of genes found significant by t-test, which may be contributing to its superior specificity relative to the other secretome signatures. The misclassification accuracies of SM1, SM2 and SM4 were compromised by expression changes of genes in one or more blood-borne diseases. Malaria (28%) and S. aureus (19%) infected patients were misclassified by SM1 as FPs (with 0.5% and 6.1% FP in controls, respectively), indicating that SM1 accuracy was affected by these underlying infections. SM4 accuracy was also impacted by S. aureus infection.

While not as sensitive as the radiation models from Zhao et al. (2018a), SM3 and SM5 exhibited high specificity for radiation (low false positivity for confounders), but less sensitive than M1-M4 and KM3-KM7. It should be feasible to accurately identify radiation exposed individuals with high sensitivity and specificity using a sequential strategy that first evaluates blood samples with suspected radiation exposures with signatures known to exhibit high sensitivity (e.g., M4), followed by identification of FPs among predicted positives with SM3 and/or SM5. This would identify and remove the misclassified, unirradiated samples, resulting in only samples that had actually received significant exposures.

While the disclosure has been described with reference to exemplary embodiments, it will be understood by those skilled in the art that various changes may be made, and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

### REFERENCES

Anderson F A Jr, Wheeler H B, Goldberg R J, Hosmer D W, Patwardhan N A, Jovanovic B, Forcier A, Dalen J E. 1991. A population-based perspective of the hospital incidence and case-fatality rates of deep vein thrombosis and pulmonary embolism. The Worcester DVT Study. Arch Intern Med. 151(5):933-8.
Bagchee-Clark A J, Mucaki E J, Whitehead T, Rogan P K. 2020. Pathway-extended gene expression signatures integrate novel biomarkers that improve predictions of patient responses to kinase inhibitors. MedComm. 1:311-327.
Banáth J P, Macphail S H, Olive P L. 2004. Radiation sensitivity, H2AX phosphorylation, and kinetics of repair of DNA strand breaks in irradiated cervical cancer cell lines. Cancer Res. 64(19):7144-7149.
Banchereau R, Jordan-Villegas A, Ardura M, Mejias A, Baldwin N, Xu H, Saye E, Rossello-Urgell J, Nguyen P, Blankenship D, et al. 2012. Host immune transcriptional profiles reflect the variability in clinical disease manifestations in patients with Staphylococcus aureus infections. PLoS One. 7(4):e34390.
Barrett A, Jacobs A, Kohn J, Raymond J, Powles R L. 1982. Changes in serum amylase and its isoenzymes after whole body irradiation. Br Med J (Clin Res Ed) 285:170-171.
Beetstra S, Thomas P, Salisbury C, Turner J, Fenech M. 2005. Folic acid deficiency increases chromosomal instability, chromosome 21 aneuploidy and sensitivity to radiation-induced micronuclei. Mutat Res. 578(1-2):317-326.
Bertho J M, Demarquay C, Frick J, Joubert C, Arenales S, Jacquet N, Sorokine-Durm I, Chau Q, Lopez M, Aigueperse J, et al. 2001. Level of Flt3-ligand in plasma: a possible new bio-indicator for radiation-induced aplasia. Int J Radiat Biol. 77(6):703-12.
Boldrini L, Bibault J E, Masciocchi C, Shen Y, Bittner M I. 2019. Deep Learning: A Review for the Radiation Oncologist. Front Oncol. 9:977.
Boldt S, Knops K, Kriehuber R, Wolkenhauer 0.2012. A frequency-based gene selection method to identify robust biomarkers for radiation dose prediction. Int J Radiat Biol. 88(3):267-76.
Braunstein S, Badura M L, Xi Q, Formenti S C, Schneider R J. 2009. Regulation of Protein Synthesis by Ionizing Radiation. Mol. Cell. Biol. 29: 5645-56.
Budworth H, Snijders A M, Marchetti F, Mannion B, Bhatnagar S, Kwoh E, Tan Y, Wang S X, Blakely W F, Coleman M, et al. 2012. DNA repair and cell cycle biomarkers of radiation exposure and inflammation stress in human blood. PLoS One. 7(11):e48619.
Collison, J. 2019. Preventing NETosis to reduce thrombosis. Nat Rev Rheumatol. 15:317.
Cruz-Garcia L, O'Brien G, Donovan E, Gothard L, Boyle S, Laval A, Testard I, Ponge L, Woźniak G, Miszczyk L, et al. 2018. Influence of Confounding Factors on Radiation Dose Estimation Using In Vivo Validated Transcriptional Biomarkers. Health Phys. 115(1):90-101.
Delgado-Rizo V, Martinez-Guzmán M A, Iñiguez-Gutierrez L, Garcia-Orozco A, Alvarado-Navarro A, Fafutis-Morris M. 2017. Neutrophil Extracellular Traps and Its Implications in Inflammation: An Overview. Front Immunol. 8:81.
Demers M, Wagner D D. 2014. NETosis: a new factor in tumor progression and cancer-associated thrombosis. Semin Thromb Hemost. 40(3):277-283.
Ding C, Peng H. 2005. Minimum redundancy feature selection from microarray gene expression data. J Bioinform Comput Biol. 3(2): 185-205.
Ding L H, Park S, Peyton M, Girard L, Xie Y, Minna J D, Story M D. 2013. Distinct transcriptome profiles identified in normal human bronchial epithelial cells after exposure to γ-rays and different elemental particles of high Z and energy. BMC Genomics. 14:372.
Disease Burden of Influenza. 2021. Centers for Disease Control and Prevention, National Center for Immunization and Respiratory Diseases (NCIRD). [accessed 2021 Apr. 9]. https://www.cdc.gov/flu/about/burden
Dorman, S N, Baranova K, Knoll J H M, Urquhart B L, Mariani G, Carcangiu M L, Rogan P K. 2016. Genomic signatures for paclitaxel and gemcitabine resistance in breast cancer derived by machine learning. Molecular oncology, 10(1), 85-100.
Dressman H K, Muramoto G G, Chao N J, Meadows S, Marshall D, Ginsburg G S, Nevins J R, Chute J P. 2007. Gene expression signatures that predict radiation exposure in mice and humans. PLoS Med. 4(4):e106.
El Atrouni W I, Knoll B M, Lahr B D, Eckel-Passow J E, Sia I G, Baddour L M. 2009. Temporal trends in the incidence of Staphylococcus aureus bacteremia in Olmsted County, Minn., 1998 to 2005: a population-based study. Clin Infect Dis. 49(12):e130-8.
Fenech M. 2010. The lymphocyte cytokinesis-block micronucleus cytome assay and its application in radiation biodosimetry. Health Phys. 98(2):234-243.
Fenech M, Knasmueller S, Bolognesi C, Bonassi S, Holland N, Migliore L, Palitti F, Natarajan A T, Kirsch-Volders M. 2016. Molecular mechanisms by which in vivo exposure to exogenous chemical genotoxic agents can lead to micronucleus formation in lymphocytes in vivo and ex vivo in humans. Mutat Res. 770(Pt A):12-25.
Ghandhi S A, Smilenov L B, Elliston C D, Chowdhury M, Amundson S A. 2015. Radiation dose-rate effects on gene expression for human biodosimetry. BMC Med Genomics. 8:22.
Goodfellow I J, Pouget-Abadie J, Mirza M, Xu B, Warde-Farley D, Ozair S, Courville A, Bengio Y. 2014. Generative adversarial nets. arxiv:1406.2661.
Greenbaum D, Luscombe N M, Jansen R, Qian J, Gerstein M. 2001. Interrelating different types of genomic data, from proteome to secretome: 'oming in on function. Genome Res. 11: 1463-1468.
Greenbaum D, Jansen R, Gerstein M. 2002. Analysis of mRNA expression and protein abundance data: an approach for the comparison of the enrichment of features in the cellular population of proteins and transcripts. Bioinformatics. 18: 585-596.
Guy J B, Bertoletti L, Magne N, Rancoule C, Mahe I, Font C, Sanz O, Martin-Antorán J M, Pace F, Vela J R, et al. 2017. Venous thromboembolism in radiation therapy cancer patients: Findings from the RIETE registry. Crit Rev Oncol Hematol. 113:83-89.
Hall J, Jeggo P A, West C, Gomolka M, Quintens R, Badie C, Laurent O, Aerts A, Anastasov N, Azimzadeh O, et al. 2017. Ionizing radiation biomarkers in epidemiological studies—An update. Mutat Res. 771:59-84.
Hassell K L. 2010. Population estimates of sickle cell disease in the U.S. Am J Prev Med. 38(45):5512-5521.
He Y, Yang F Y, Sun E W. 2018. Neutrophil Extracellular Traps in Autoimmune Diseases. Chin Med J (Engl). 131(13):1513-1519.
Hill A, Hanson M, Bogle M A, Duvic M. 2004. Severe radiation dermatitis is related to Staphylococcus aureus. Am J Clin Oncol. 27(4):361-363.
Hounkpe B W, Chenou F, Domingos I F, Cardoso E C, Costa Sobreira M J V, Araujo A S, Lucena-Araújo A R, da Silva Neto P V, Malheiro A, Fraiji N A, et al. 2020. Neutrophil extracellular trap regulators in sickle cell disease: Modulation of gene expression of PADI4, neutrophil elastase, and myeloperoxidase during vaso-occlusive crisis. Res Pract Thromb Haemost. 16; 5(1):204-210.
Jacobs A R, Guyon T, Headley V, Nair M, Ricketts W, Gray G, Wong J Y C, Chao N, Terbrueggen R. 2020. Role of a high throughput biodosimetry test in treatment prioritization after a nuclear incident. Int J Radiat Biol. 96(1):57-66.
Jen K Y, Cheung V G. 2003. Transcriptional response of lymphoblastoid cells to ionizing radiation. Genome Res. 13(9):2092-100.
Kirsch-Volders M, Plas G, Elhajouji A, Lukamowicz M, Gonzalez L, Vande Loock K, Decordier I. 2011. The in vitro M N assay in 2011: origin and fate, biological significance, protocols, high throughput methodologies and toxicological relevance. Arch Toxicol. 85(8):873-99.
Kirsch-Volders M, Fenech M, Bolognesi C. 2018. Validity of the Lymphocyte Cytokinesis-Block Micronucleus Assay (L-CBMN) as biomarker for human exposure to chemicals with different modes of action: A synthesis of systematic reviews. Mutat Res Genet Toxicol Environ Mutagen. 836(Pt A):47-52.
Knops K, Boldt S, Wolkenhauer O, Kriehuber R. 2012. Gene expression in low- and high-dose-irradiated human peripheral blood lymphocytes: possible applications for biodosimetry. Radiat Res. 178(4):304-12.
Lewis D A, Stashenko G J, Akay O M, Price L I, Owzar K, Ginsburg G S, Chi J T, Ortel T L. 2011. Whole blood gene expression analyses in patients with single versus recurrent venous thromboembolism. Thromb Res. 128(6):536-40.
Li Y, Shirley B C, Wilkins R C, Norton F, Knoll J H M, Rogan P K. 2019. Radiation dose estimation by completely automated interpretation of the dicentric chromosome assay. Rad. Protect. Dosim. 186(1): 42-47.
Liu J, Li Y, Wilkins R, Flegal F, Knoll J H M, Rogan P K. 2017. Accurate cytogenetic biodosimetry through automated dicentric chromosome curation and metaphase cell selection [version 1; peer review: 2 approved]. F1000Res. 6:1396.
Lu T P, Hsu Y Y, Lai L C, Tsai M H, Chuang E Y. 2014. Identification of gene expression biomarkers for predicting radiation exposure. Sci Rep. 4:6293.
Mah L J, E I-Osta A, Karagiannis T C. 2010. gammaH2AX: a sensitive molecular marker of DNA damage and repair. Leukemia. 24(4):679-686.
Meadows S K, Dressman H K, Muramoto G G, Himburg H, Salter A, Wei Z, Ginsburg G S, Chao N J, Nevins J R, Chute J P. 2008. Gene expression signatures of radiation response are specific, durable and accurate in mice and humans. PLoS One. 3(4):e1912.
Mucaki E J, Baranova K, Pham H Q, Rezaeian I, Angelov D, Ngom A, Rueda L, Rogan P K. 2016. Predicting Outcomes of Hormone and Chemotherapy in the Molecular Taxonomy of Breast Cancer International Consortium (METABRIC) Study by Biochemically-inspired Machine Learning [version 3; peer review: 2 approved]. F1000Research. 5:2124.
Mucaki E J, Zhao J, Lizotte D J, Rogan P K. 2019. Predicting responses to platin chemotherapy agents with biochemically-inspired machine learning. Signal transduction and targeted therapy. 4:1.
Mucaki E J, Rogan P K. 2021. Zenodo Archive for “Improved radiation gene expression profiles with sequentially applied, sensitive and specific gene signatures”. Zenodo. https://doi.org/10.5281/zenodo.5009008
Nallandhighal S, Park G S, Ho Y Y, Opoka R O, John C C, Tran T M. 2019. Whole-Blood Transcriptional Signatures Composed of Erythropoietic and NRF2-Regulated Genes Differ Between Cerebral Malaria and Severe Malarial Anemia. J Infect Dis. 219(1):154-164.
Oh D S, Cheang M C, Fan C, Perou C M. 2014. Radiation-induced gene signature predicts pathologic complete response to neoadjuvant chemotherapy in breast cancer patients. Radiat Res. 181(2):193-207.
Ostheim P, Don Mallawaratchy A, Müller T, Schüle S, Hermann C, Popp T, Eder S, Combs S E, Port M, Abend M. 2021. Acute radiation syndrome-related gene expression in irradiated peripheral blood cell populations. Int J Radiat Biol. 97(4):474-484.
Paul S, Amundson S A. 2008. Development of gene expression signatures for practical radiation biodosimetry. Int J Radiat Oncol Biol Phys. 71(4):1236-1244.
Paul S, Amundson S A. 2011. Gene expression signatures of radiation exposure in peripheral white blood cells of smokers and non-smokers. Int J Radiat Biol. 87(8):791-801.
Pernot E, Hall J, Baatout S, Benotmane M A, Blanchardon E, Bouffler S, El Saghire H, Gomolka M, Guertler A, Harms-Ringdahl M, et al. 2012. Ionizing radiation biomarkers for potential use in epidemiological studies. Mutat Res. 751(2):258-286.
Port M, Hérodin F, Valente M, Drouet M, Lamkowski A, Majewski M, Abend M. 2017. Gene expression signature for early prediction of late occurring pancytopenia in irradiated baboons. Ann Hematol. 96(5):859-870.
Qi J-L, He J-R, Liu C-B, Jin S-M, Gao R-Y, Yang X, Bai H-M, Ma Y-B. 2020. Pulmonary Staphylococcus aureus infection regulates breast cancer cell metastasis via neutrophil extracellular traps (NETs) formation. MedComm. 1:188-201.
Quinlan J, Idaghdour Y, Goulet J P, Gbeha E, de Malliard T, Bruat V, Grenier J C, Gomez S, Sanni A, Rahimy M C, Awadalla P. 2014. Genomic architecture of sickle cell disease in West African children. Front Genet. 5:26.
Rhee Y, Aroutcheva A, Hota B, Weinstein R A, Popovich K J. 2015. Evolving Epidemiology of Staphylococcus aureus Bacteremia. Infect Control Hosp Epidemiol. 36(12):1417-22.
Rieger K E, Hong W J, Tusher V G, Tang J, Tibshirani R, Chu G. 2004. Toxicity from radiation therapy associated with abnormal transcriptional responses to DNA damage. Proc Natl Acad Sci USA. 101(17):6635-40.
Risques R A, Lai L A, Brentnall T A, Li L, Feng Z, Gallaher J, Mandelson M T, Potter J D, Bronner M P, Rabinovitch P S. 2008. Ulcerative colitis is a disease of accelerated colon aging: evidence from telomere attrition and DNA damage. Gastroenterology. 135(2):410-8.
Rogan P K, Li Y, Wilkins R C, Flegal F N, Knoll J H. 2016. Radiation Dose Estimation by Automated Cytogenetic Biodosimetry. Radiat Prot Dosimetry. 172(1-3):207-217.
Rogan P K. 2019. Multigene signatures of responses to chemotherapy derived by biochemically-inspired machine learning. Mol Genet Metab. 128(1-2):45-52.
Rogan P K, Mucaki E J, Shirley B C. 2020. Characteristics of human and viral RNA binding sites and site clusters recognized by SRSF1 and RNPS1. Zenodo. http://www.doi.org/10.5281/zenodo.3737089
Rogan P K, Mucaki E J and Shirley B C. 2021. A proposed molecular mechanism for pathogenesis of severe RNA-viral pulmonary infections [version 2; peer review: 4 approved]. F1000Research. 9:943.
Sedelnikova O A, Bonner W M. 2006. GammaH2AX in cancer cells: a potential biomarker for cancer diagnostics, prediction and recurrence. Cell Cycle. 5:2909-2913.
Sharif R, Thomas P, Zalewski P, Fenech M. 2012. Zinc deficiency or excess within the physiological range increases genome instability and cytotoxicity, respectively, in human oral keratinocyte cells. Genes Nutr. 7(2):139-154.
Shirley B, Li Y, Knoll J H M, Rogan P K. 2017. Expedited Radiation Biodosimetry by Automated Dicentric Chromosome Identification (ADCI) and Dose Estimation. J Vis Exp. (127):56245.
Shirley B C, Knoll J H M, Moquet J, Ainsbury E, Pham N D, Norton F, Wilkins R C, Rogan P K. 2020. Estimating partial-body ionizing radiation exposure by automated cytogenetic biodosimetry. Int J Radiat Biol. 96(11):1492-1503.
Song Y, Chung C S, Bruno R S, Traber M G, Brown K H, King J C, Ho E. 2009. Dietary zinc restriction and repletion affects DNA integrity in healthy men. Am J Clin Nutr. 90(2):321-8.
Spivak J L, Considine M, Williams D M, Talbot C C Jr, Rogers O, Moliterno A R, Jie C, Ochs M F. 2014. Two clinical phenotypes in polycythemia vera. N Engl J Med. 371(9):808-17.
Tapio S. 2013. Ionizing Radiation Effects on Cells, Organelles and Tissues on Proteome Level. pp 37-48 In: Leszczynski D. (eds) Radiation Proteomics. Advances in Experimental Medicine and Biology, vol 990. Springer, Dordrecht.
Tichy A, Kabacik S, O'Brien G, Pejchal J, Sinkorova Z, Kmochova A, Sirak I, Malkova A, Beltran C G, Gonzalez J R, et al. 2018. The first in vivo multiparametric comparison of different radiation exposure biomarkers in human blood. PLoS One. 13(2):e0193412.
Vanderwerf S M, Svahn J, Olson S, Rathbun R K, Harrington C, Yates J, Keeble W, Anderson D C, Anur P, Pereira N F, et al. 2009. TLR8-dependent TNF-(alpha) overexpression in Fanconi anemia group C cells. Blood. 114(26):5290-8.
Wang Q, Lee Y, Shuryak I, Pujol Canadell M, Taveras M, Perrier J R, Bacon B A, Rodrigues M A, Kowalski R, Capaccio C, et al. 2020. Development of the FAST-DOSE assay system for high-throughput biodosimetry and radiation triage. Sci Rep. 10(1):12716.
Warters R L, Adamson P J, Pond C D, Leachman S A. 2005. Melanoma cells express elevated levels of phosphorylated histone H2AX foci. J Invest Dermatol. 124:807-817.
Yu T, MacPhail S H, Banath J P, Klokov D, Olive P L. 2006. Endogenous expression of phosphorylated histone H2AX in tumors in relation to DNA double-strand breaks and genomic instability. DNA Repair (Amst). 5:935-946.
Zeng Z, Zhan J, Chen L, Chen H, Cheng S. 2021. Global, regional, and national dengue burden from 1990 to 2017: A systematic analysis based on the global burden of disease study 2017. E Clinical Medicine. 32:100712.
Zhao J Z L, Mucaki E J Rogan P K. 2018a. Predicting ionizing radiation exposure using biochemically-inspired genomic machine learning [version 2; peer review: 3 approved]. F1000Research. 7:233.
Zhao J Z L, Mucaki E J, Rogan P K. 2018b. Matlab Code for “Predicting Exposure to Ionizing Radiation by Biochemically-Inspired Genomic Machine Learning”. Zenodo. https://doi.org/10.5281/zenodo.1170571

What is claimed is:

1. A method for determining radiation gene expression profile of a sample, comprising the steps of:
a) providing a sample of target cells from a sample of an individual;
b) evaluating the sample of target cells for radiation exposure with a first gene signature;
c) detecting radiation exposure with gene signatures, wherein said first signature is a highly sensitive radiation gene signature;
d) evaluating the sample of target cells against a second gene signature, wherein the second signature is a radiation gene signature with high specificity, and
e) using the second gene signature in step D to identify and remove any misclassified unirradiated samples remaining after evaluating all samples indicated as irradiated using the gene signature obtained in step c).

2. The method of claim 1, wherein said first gene signature includes one of the signatures designated as either M1, M2, M3, M4, KM1, KM2, KM4, KM6, or KM7.

3. The method of claim 1, wherein said second gene signature includes the signature designated as either SM3 or SM5.

4. The method of claim 1, further comprising,
rejecting radiation signatures with high false positive rates in confounding conditions.

5. The method of claim 1, further comprising,
deriving radiation signatures with low misclassification rates in confounders in both controls and test samples.

6. The method of claim 1 further comprising,
mitigating false positive predictions due to differential expression caused by confounding conditions by sequentially evaluating both the first gene signature and the second signature.

7. The method of claim 1, wherein the selection of the second gene signature minimizes inclusion of genes, gene products or genes in the same biochemical pathways with gene expression changes that are common to both radiation exposed and any population of individuals with confounding phenotypes or diagnoses.

8. The method of claim 7, where removal of one or more genes from the first signature reduces misclassification of unirradiated samples using a highly sensitive gene signature.

9. A method for determining a radiation gene expression profile, comprising the steps of:
a) providing a sample of target cells from a sample of an individual;
b) evaluating the sample of target cells for radiation exposure with gene signatures;
c) detecting radiation exposure in the sample of target cells with a first gene signature, wherein the first gene signature is a highly sensitive radiation gene signature;
d) evaluating the sample of cells against a second gene signature, wherein the second signature is a high specificity radiation gene signature, and
e) using the second gene signature from step d) to identify if the sample is a misclassified unirradiated sample from step c),
f) repeating steps a) through e) with additional samples, and removing all misclassified samples identified.

10. The method of claim 9, wherein the first gene signature includes one of the signatures designated M1, M2, M3, M4, KM1, KM2, KM4, KM6, or KM7, and the second gene signature includes either of the signatures designated SM3 or SM5.

11. A method for determining radiation gene expression profiles, comprising the steps of:
a. providing a sample of target cells from a sample from an individual;
b. evaluating the sample of target cells for radiation exposure with a gene signature;
c. detecting radiation exposure in the sample of target cells using a gene signature, wherein the signature is both highly sensitive and highly specific for radiation.

12. The method of claim 11, wherein the gene signature includes either of the signatures designated KM3 and KM5.

13. A method for determining radiation gene expression profiles, comprising the steps of:
a) providing a sample of target cells from a patient;
b) evaluating the sample of target cells for radiation exposure with a first gene signature;
c) detecting radiation exposure with a first gene signature and a second gene signature, wherein the first signature is a highly sensitive radiation gene signature;
d) evaluating the sample against the second gene signature, wherein the second signature is a radiation gene signature with high specificity, and
e) determining if the sample is an unirradiated sample misclassified as irradiated with the gene signatures obtained in step c), wherein the first gene signature includes either of the signatures designated as one of M1, M2, M3, M4, KM1, KM2, KM4, KM6, or KM7, and the second gene signature includes either of the signatures designated as SM3 or SM5,
f) repeating steps a) through e) on additional samples, and removing all misclassified samples identified.

14. The method of claim 1, said method being used to evaluate environmental or biomedical radiation exposures to an individual.

15. The method of claim 14, said method being used to evaluate clinically relevant radiation exposure.

16. The method of claim 11, said method being used to evaluate environmental or biomedical radiation exposures to an individual.

17. The method of claim 16, said method being used to evaluate clinically relevant exposure.
